(19) United States
(12) Patent Application Publication
(10) Pub. No.: US 2025/0169846 A1
(43) Pub. Date: May 29, 2025

(54) TIP FOR NEUROSURGICAL ROBOT

(71) Applicant: Tamar Robotics LTD, Kibbutz Yagur (IL)

(72) Inventors: Noam HASSIDOV, Moshav Bustan HaGalil (IL); Hadas ZISO, Kiryat-Tivon (IL); Tom SHTENDEL, Kiryat-Tivon (IL); Ofri GLIKSMAN, Rosh Pina (IL); Dan BLECHER, Ramat-Gan (IL)

(73) Assignee: Tamar Robotics LTD, Kibbutz Yagur (IL)

(21) Appl. No.: 18/839,774

(22) PCT Filed: Feb. 23, 2023
(86) PCT No.: PCT/IL2023/050186

§ 371 Date: Aug. 20, 2024

Related U.S. Application Data
(60) Provisional application No. 63/312,894, filed on Feb. 23, 2022.

(51) Int. Cl. A61B 17/3203

(52) U.S. Cl. CPC A61B 17/3203

(57) ABSTRACT

Device for selective tissue removal. In some embodiments, a tip of the device includes a housing, a nozzle positioned to jet fluid supplied to the nozzle as a fluid jet disposed within and restricted by a volume defined by the housing, and an opening in the housing to the fluid jet, sized and shaped for the fluid jet to abrade tissue adjacent to said volume.

### RELATED APPLICATIONS

This application claims the benefit of priority under 35 USC § 119(c) of U.S. Provisional Patent Application No. 63/312,894, filed Feb. 23, 2022, the contents of which are incorporated herein by reference in their entirety.

### FIELD AND BACKGROUND OF THE INVENTION

The present invention, in some embodiments thereof, relates to the field of tissue extraction and, more particularly, but not exclusively, tissue extraction by fluid jet tissue erosion.

### Background Art Includes

U.S. Pat. No. 10,980,669, which discloses “A water jet instrument may be used for manually performing surgery. The water jet instrument may be manually controlled or controlled by a system with a robotic control. The water jet apparatus defines a jet cutting area that is based at least in part on a flow rate meter and a feedback loop.”

U.S. Patent Application Publication No. US 2021/0298954, which discloses “A water jet system may be used for performing surgery. The water jet system includes a water jet device to provide a water jet flow to break apart tissue in an enclosed operative site. The water jet system includes an aspiration device to remove material from the enclosed operative site. The water jet system includes a control system with an aspiration pump coupled to the aspiration device and to draw material through the aspiration device. The control system includes an aspiration flow rate meter to measure an aspiration flow rate through the aspiration pump. The control system includes a controller to control operation of the aspiration pump based at least in part on feedback from the aspiration flow meter. The control system is configured to provide a targeted aspiration flow.”

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A device for selective tissue removal comprising a tip having: a housing sized and shaped to enter a cranial cavity through an aperture 5 mm in diameter; a nozzle positioned to jet fluid supplied to the nozzle as a fluid jet disposed within and restricted by a volume defined by the housing; and an opening in the housing to the fluid jet, sized and shaped for the fluid jet to erode tissue adjacent to the volume.

Example 2. The device according to example 1, wherein the opening is sized and shaped to allow the tissue to intrude into the opening.

Example 3. The device according to any one of examples 1-2, wherein the opening has a height of 0.5-2 mm.

Example 4. The device according to example 3, wherein the tip extends in a proximal-distal direction and wherein the height is measured in the proximal-distal direction.

Example 5. The device according to example 1, wherein the opening is to a side of the fluid jet.

Example 6. The device according to any one of examples 1-5, wherein a portion of the fluid jet flows alongside the opening.

Example 7. The device according to any one of examples 1-6, wherein the jet impinges on an inner surface of the housing.

Example 8. The device according to any one of examples 1-7, wherein the fluid jet is in a direction alongside or away from the opening, the fluid jet not impinging directly onto tissue outside the housing.

Example 9. The device according to any one of examples 1-8, wherein the tip comprises a nozzle and a blocker, wherein the fluid jet is jetted through the nozzle and travel of the fluid jet is restricted by the blocker.

Example 10. The device according to example 9, wherein the tip has a proximal and a distal end, the blocker is positioned distal of the nozzle, and disrupts flow of jetted fluid distal of the blocker.

Example 11. The device according to example 10, wherein the blocker prevents flow of the fluid jet distal of the blocker.

Example 12. The device according to any one of examples 10-11, wherein the fluid flow from the nozzle is in a direction including a proximal-distal component.

Example 13. The device according to any one of examples 1-8, wherein the tip has a proximal and a distal end and, where the fluid jet flows in a direction including a proximal-distal component.

Example 14. The device according to any one of examples 1-8, or 13, wherein the fluid jet is formed by passing fluid through a nozzle which is disposed distal of the opening.

Example 15. The device according to any one of examples 13-14, comprising a fluid supply conduit which supplies fluid to the nozzle, wherein the fluid supply conduit extends in a proximal-distal direction and includes a turn at the tip prior to fluidly connect to the nozzle to supply fluid for the fluid jet.

Example 16. The device according to example 15, wherein the device comprises a suction conduit having suction inlet disposed at the tip for extraction of fluid and eroded tissue.

Example 17. The device according to any one of examples 1-16, wherein the device comprises an arm hosting the tip at a distal portion thereof.

Example 18. The device according to example 17, wherein the arm is bendable at one or more joint.

Example 19. The device according to example 18, wherein the device includes one or more actuator configured to bend the arm at the one or more joint.

Example 20. The device according to any one of examples 17-19, wherein the device comprises a suction conduit having suction inlet disposed at the tip for extraction of fluid and eroded tissue; and wherein the fluid supply conduit and the suction conduit extend through the arm.

Example 21. The device according to example 20, wherein the fluid supply conduit extends through a lumen of the suction conduit.

Example 22. The device according to any one of examples 1-21, comprising a second nozzle positioned to jet fluid supplied to the second nozzle as a second fluid jet disposed within and restricted by the volume defined by the housing.

Example 23. The device according to example 22, wherein the housing comprises a second opening, sized and shaped for the second fluid jet to erode tissue adjacent to the volume.

Example 24. The device according to any one of examples 1-23, comprising a sensor configured to sense one or more fluid parameter of fluid supplied to the nozzle.

Example 25. The device according to any one of examples 1-24 wherein the tissue is tissue within a cranial cavity.

Example 26. A method of removal of target tissue comprising: positioning an opening to a housing adjacent to the target tissue, which housing hosts an outlet to a fluid supply; and supplying fluid to the outlet to provide a fluid flow within the housing which fluid flow interacts, through the opening, with the target tissue to erode the target tissue.

Example 27. The method according to example 26, wherein the outlet comprises a nozzle, which produces a fluid jet upon the supplying.

Example 28. The method according to example 27, wherein the fluid jet is at least partially enclosed by the housing.

Example 29. The method according to example 28, wherein the housing encloses 10-90% of a cross sectional circumference of the fluid jet.

Example 30. The method according to any one of examples 27-28, comprising blocking a path of the water jet.

Example 31. The method according to any one of examples 26-30, comprising removing eroded tissue.

Example 32. The method according to example 31, wherein the removing is by suctioning eroded tissue and jetted fluid.

Example 33. The method according to any one of examples 26-32, where the eroding is of a portion of the target, and the method comprises repositioning the opening to continue to erode target tissue.

Example 34. The method according to any one of examples 26-33, wherein the target tissue is tissue within a cranial cavity.

Example 35. The method according to example 34, wherein the positioning comprises accessing a region within a cranial cavity.

Example 36. A device for selective tissue removal comprising: a tip comprising: an outlet to a fluid supply conduit fluid exiting the outlet extending in a first direction; and a blocker extending transverse to the first direction to intercept the fluid exiting the outlet.

Example 37: A device for selective tissue removal comprising: an arm having a fluid supply conduit and extending in a proximal-distal direction; and a tip disposed at a distal end of the arm, the tip having: a housing, an opening in the housing, and an outlet to the fluid supply conduit distal of the opening, the opening disposed to supply fluid into and/or across the opening.

Further examples include:

Example 38. A device for selective tissue removal comprising: a tip comprising: a housing; a nozzle positioned to jet fluid supplied to the nozzle as a fluid jet disposed within and restricted by a volume defined by the housing; and an opening in the housing to the fluid jet, sized and shaped for the fluid jet to abrade tissue adjacent to the volume.

Example 39. The device according to example 22, wherein the housing comprises a second opening, sized and shaped for the second fluid jet to abrade tissue adjacent to the volume.

Example 40. A device for selective tissue removal comprising: an arm comprising a fluid supply conduit and extending in a proximal-distal direction and comprising a tip disposed at a distal end of the arm, the tip comprising: a housing; an opening in the housing; and an outlet to the fluid supply conduit distal of the opening, the opening disposed to supply fluid into and/or across the opening.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Instruction executing elements of the processor may comprise, for example, one or more microprocessor chips, ASICs, and/or FPGAs. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. Additionally or alternatively, sequences of logical operations (optionally logical operations corresponding to computer instructions) may be embedded in the design of an ASIC and/or in the configuration of an FPGA device. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 is a simplified schematic of a system, according to some embodiments of the present disclosure;

FIG. 2 is a method of tissue removal, according to some embodiments of the present disclosure;

FIGS. 3A-3D are simplified schematics illustrating removal of target tissue, using a tip, according to some embodiments of the present disclosure;

FIG. 3E is a simplified schematic illustrating removal of target tissue, using a tip, according to some embodiments of the present disclosure;

FIG. 4 is a flow chart of a method of treatment, according to some embodiments of the present disclosure;

FIGS. 5A-5B are simplified schematics illustrating removal of target tissue, according to some embodiments of the present disclosure;

FIG. 6A is a simplified schematic cross section of a tip, according to some embodiments of the present disclosure;

FIG. 6B is a simplified schematic sectional view of a tip, according to some embodiments of the present disclosure;

FIG. 7 is a simplified schematic cross section of a tip, according to some embodiments of the present disclosure;

FIG. 8 is a simplified schematic cross section of a tip, according to some embodiments of the present disclosure;

FIG. 9A is a simplified schematic of a portion of an arm including a tip, according to some embodiments of the present disclosure;

FIG. 9B is a simplified schematic sectional view of a tip, according to some embodiments of the present disclosure;

FIG. 10 is a simplified schematic of a tip, according to some embodiments of the present disclosure;

FIG. 11 is a simplified schematic of a tip, according to some embodiments of the present disclosure;

FIG. 12 is a simplified schematic of a tip, according to some embodiments of the present disclosure;

FIG. 13A is a simplified schematic of a portion of an arm including a tip, according to some embodiments of the present disclosure;

FIG. 13B is a simplified schematic section view of a portion of an arm including a tip, according to some embodiments of the present disclosure;

FIG. 13C is a simplified schematic of a portion of an arm including a tip, according to some embodiments of the present disclosure;

FIG. 14 is a simplified schematic section view of a tip, according to some embodiments of the present disclosure;

FIG. 15 is a simplified schematic of a tip, according to some embodiments of the present disclosure;

FIG. 16 is a simplified schematic of a tip, according to some embodiments of the present disclosure;

FIG. 17 is a simplified schematic of a portion of an arm including a tip, according to some embodiments of the present disclosure;

FIG. 18 is a simplified schematic of a portion of a tip, according to some embodiments of the present disclosure;

FIG. 19 is a simplified schematic of a portion of an arm including a tip, according to some embodiments of the present disclosure;

FIG. 20 is a simplified schematic of a tip, according to some embodiments of the present disclosure; and

FIGS. 21A-21C are simplified schematic sectional views of nozzles, according to some embodiments of the present disclosure.

### DESCRIPTION OF SPECIFIC EMBODIMENTS OF THE INVENTION

The present invention, in some embodiments thereof, relates to the field of tissue extraction and, more particularly, but not exclusively, tissue extraction by fluid jet tissue erosion.

### Overview

A broad aspect of some embodiments of the present disclosure relates to removal of target tissue using a restricted cutting zone. A potential benefit being accurate removal of tissue, with minimal damage to surrounding tissue. In some embodiments, the cutting zone is provided by a fluid jet. Optionally, a restricted portion of the fluid jet is used to erode target tissue.

An aspect of some embodiments of the present disclosure relates to erosion of tissue by a fluid jet, wherein erosion occurs at a tissue-fluid interface at side region(s) of the jet. In some embodiments, side region(s) include those in a direction from the jet not perpendicular to a direction of jetting of the fluid jet.

In an exemplary embodiment, the tissue to be eroded is brain tissue and/or tissue within a cranial cavity. In some embodiments, the tip enters the cranial cavity through an aperture 10 mm in diameter or less. In some embodiments, the tip is sized and shaped to enter the cranial cavity through an aperture having a diameter of between 0.5-10 mm, between 0.5-5 mm, between 0.5-2 mm, or a diameter in another range. In some embodiments, entry into the cranial cavity comprises penetration through a hole made in bone surrounding the cranial cavity.

In some embodiments, the fluid of the fluid jet includes saline and/or water and/or cerebrospinal fluid (CSF). In some embodiments, cerebrospinal fluid is extracted from a subject and then used as a supply to the fluid jet. In some embodiments, removed debris and fluid is filtered to remove debris before being re-supplied as jetting fluid.

In some embodiments, the fluid jet is at least partially enclosed; e.g., enclosed within a housing. Optionally, interaction between the fluid jet and tissue to be eroded is through one or more openings (also herein termed “gaps” and “spaces”) in the housing allowing access to the jet. Alternatively or additionally, erosion occurs in a space across which fluid is jetted before extraction through an extraction conduit; e.g., a space between the outlet of a supply conduit and an extraction conduit inlet, wherein the opening and/or space is sized and/or shaped to allow jetted fluid to exit therefrom and/or allow tissue to enter inwards to be accessed and/or eroded by the fluid jet. In some embodiments, the tissue is eroded by abrasion of the jet against the tissue.

In some embodiments, the water jet does not directly impinge on tissue. For example, a blocker prevents travel in a direction of jetting of at least a portion of the jetted fluid. In some embodiments, the blocker is part of and/or is attached to the housing enclosing side(s) of the fluid jet. In some embodiments, the water jet does not extend beyond the tip and/or a distal end of the tip.

In some embodiments, a proportion of a circumference of sides of the water jet is potentially allowed to enable with tissue, wherein a proportion of the circumference is blocked from interaction; e.g., enclosed by the housing. For example, the tissue-jetted water interface is a proportion of the circumference, e.g., 10-90%, or 20-80%, or 30-90%, or lower or higher or intermediate ranges or percentages, of the circumference. In some embodiments, one or more discrete portions of the circumference form the tissue-jetted water interface. For example, the housing has more than one separated opening.

In some embodiments, fluid is supplied at a distance from the opening and/or space wherein fluid interacts with tissue to be eroded; for example, within the housing and away from the opening in the housing. In some embodiments, the fluid supply conduit outlet is 0.5-5 mm, or 1-3 mm, or lower or higher intermediate distances from the opening. In some embodiments, the fluid supply conduit outlet is located at a central region of a cross section of the housing (e.g., central 10-50% of a cross section of the housing and/or tip extent—optionally as defined by the housing—or lower or higher or intermediate ranges or percentages).

In some embodiments, an extent of the tissue-jetted water interface is adjustable. For example, by changing dimension(s) of an opening of the delivery apparatus through which the jetted water interacts with adjacent tissue. For example by adjusting parameters of jetted water (e.g., pressure, flow rate, pulse duty cycle and/or phase relationship between jetting and suction for embodiments in which jetting and/or suction are provided in pulses). For example, by adjusting an extent of a jetting zone through a gap in the tip (e.g., for embodiments in which the position and/or size of the gap is adjustable in one or more directions). For example, adjusting comprises adjusting a size, shape and/or position of fluid supply outlet(s).

In some embodiments, fluid is supplied and/or suction is applied continuously.

In some embodiments, jetted fluid and/or suction is applied in pulses. In some embodiments, the pulses are in phase. In some embodiments, the pulses are antiphase, or at least partially out of phase. In some embodiments, the suction and jetting pulses have about the same duration. In some embodiments, the pulses have different durations. In some embodiments, one or both of duty cycle and frequency of pulses of fluid supply and/or suction are controlled; e.g., to control pressure (e.g., ICP) at the treatment site and/or to control erosion of tissue (e.g., rate and/or extent of erosion).

In an exemplary embodiment, fluid supply is in pulses (e.g., the duty cycle and/or amplitude of which are controllable) and suction is at a constant level (e.g., flow rate).

In some embodiments, jetted water and/or eroded tissue and/or other debris is extracted; e.g., by suction. In some embodiments, extraction is while the tip is in position at a treatment device. In some embodiments, extraction is during erosion, e.g., while jetting suction is applied. Potentially, extraction during erosion increases visibility of a treatment site, potentially improving accuracy of removal of tissue. Potentially extraction of fluid and/or eroded tissue (e.g., during erosion) prevents dispersal of eroded tissue (e.g., diseased tissue).

In some embodiments, erosion of tissue occurs, at least partially, within the tip; for example, tissue intruding into the opening(s) in the tip is eroded. In some embodiments, extraction occurs within the tip housing (e.g., suction is applied within the tip housing). In some embodiments, both erosion and extraction occur within the tip, a potential benefit being prevention of dispersal of fluid and/or eroded tissue.

In some embodiments, the water jet is provided at a tip of a tissue device arm. In some embodiments, the arm is elongate. In some embodiments, the arm includes one or more actuators for positioning portion(s) of the arm and/or tip; e.g., the arm is a robotically controlled.

In some embodiments, a direction of jetting is parallel to a direction of elongation of the arm, or within 45°, or 30°, or 20°, or 10° of parallel, or lower or higher or intermediate ranges or angles.

In some embodiments, jetting is in a proximal distal direction (e.g., a nozzle being located proximal of opening(s) in the tip housing to the jet). Optionally, a blocker prevents direct jetting of fluid distal of the blocker.

In some embodiments, fluid supplied to a treatment site is refracted in direction prior to exiting a delivery apparatus. In some embodiments, fluid is supplied to the tip in a first direction and then routed, e.g., by a supply conduit, in a direction other to said first direction (e.g., 90-180° to), for example, prior to exiting the supply apparatus.

For example, jetting is in a distal proximal direction from a fluid supply conduit directing the fluid proximally, e.g., prior to jetting through a nozzle. In some embodiments, fluid is supplied extending through and/or alongside the arm, and the fluid supply conduit is refracted; e.g., at a distal end portion thereof, e.g., prior to jetting across an opening in the tip housing.

In some embodiments, fluid and suction are applied to a treatment area by conduits coupled to each other and/or positioned together. Optionally, fluid and extraction conduits are part of and/or coupled to and/or attached to an arm. Optionally, a fluid delivery conduit passes through a lumen of a suction conduit.

In some embodiments, erosion at the fluid-tissue interface is gradual and/or the fluid-tissue interface is small, for example, with respect to a size of the target tissue (e.g., extent of the target tissue in one or more directions being at least 1.5 times, or 2 times, or 5 times, or lower or higher or intermediate multiples a dimension of an opening to the tip, in one or more directions). Optionally, an opening in the tip housing is 0.5-5 mm, or 1-4 mm, or 2-3 mm, or about 2.5 mm, or lower or higher or intermediate sizes or ranges, in one or more directions, for example, in two directions defining a plane and/or surface between which the tissue and supplied fluid interact.

In some embodiments, the tip is sufficiently rigid for the opening(s) in the housing to remain open and/or not deform; e.g., under suction pressure of extraction and/or pressure of tissue. In some embodiments, the supply conduit is refracted at the tip the conduit itself and/or a housing to the conduit is sufficiently rigid to remain open and/or not deform. In some embodiments, a rigid distal portion of a tip is short in length (e.g., less than 5 mm long, less than 3 mm long, about 2.5 mm long or lower or higher or intermediate lengths) potentially easing positioning of the tip.

In some embodiments, the tip is sufficiently rigid to be advanced through tissue, for example, during positioning of the tip at a treatment site. For example, erosion is both by the water jet and by interaction between the outside of the tip and tissue (e.g., cutting of tissue by advancing the tip through and/or alongside the tissue).

In some embodiments, the arm to which the tip is distally disposed includes one or more flexible portions and/or joints, enabling positioning of the arm and/or tip. In some embodiments, a length of the tip and/or rigid portion(s) of the tip, length measured along a central longitudinal axis of the arm is small. For example, rigid portion(s) of the tip being 0.5-10 mm, or 0.5-5 mm, or 0.5-3 mm, or 1-3 mm, or lower or higher or intermediate dimensions or ranges.

Optionally, the supply conduit is refracted at the tip, the supply conduit is small in extent. For example, having a cross sectional area defined by a diameter of less than 5 mm, or less than 3 mm, or less than 2 mm, or less than 1 mm, or lower or higher or intermediate cross sectional diameters. A potential benefit being a smaller extent rigid tip.

In some embodiments, fluid is delivered through more than one aperture (e.g., nozzle) and/or through more than one supply conduit. Optionally, in some embodiments, a treatment region is irrigated and/or suction; e.g., by another fluid source than that of the supply conduit. Optionally, additional irrigation and/or suction provides increased visibility to the treatment area.

In some embodiments, fluid is jetted through a plurality of apertures. Optionally, a plurality of smaller volume jets are more effective at rapidly but gently eroding tissue. In some embodiments, fluid is selectively jetted through a plurality of apertures; e.g., located at different locations on the tip. For example, potentially enabling a change in location of the jetting zone; e.g., without moving the tip.

In some embodiments, fluid is selectively supplied through a plurality of delivery conduits. For example, an outlet is fluidly connected to a plurality of delivery conduits supplying through a selected number of conduits is used to change fluid parameter(s). For example, flow is through a higher or lower number of conduits, in some embodiments, being relating to higher or lower jetting flow at the outlet.

In some embodiments, alternatively or additionally to fluid jet erosion, erosion of tissue is mechanical. For example, the tip cuts tissue; e.g., the tip having sharp and/or pointed edge(s). Optionally, cutting is by mechanical movement of one or more elements (e.g., with fluid jets as described herein being replaced by a cutting edge; e.g., a moving cutting edge). Optionally, fluid is supplied for irrigation and/or to aid extraction of eroded tissue and/or other debris (e.g., blood).

In some embodiments, nozzle and/or fluid flow parameters are selected to produce a fluid jet which is one or both of strong and having a well-defined edge.

In some embodiments, fluid flow supplied to the nozzle is 0.05-0.4 liters/minute, or 0.1-0.3 liters/minute, or 0.15-0.25 liters/minute, or about 0.2 liters/minute or lower or higher or intermediate ranges or flow rates.

In some embodiments, a rate of removal of material (e.g., fluid and/or eroded debris) is balanced with that of fluid supply and/or erosion. Optionally, fluid flow at the nozzle and suction are balanced to maintain a desired pressure at the treatment region.

For example, during treatment of intracranial tissue, fluid flow and/or suction are selected to control and/or affect and/or maintain intracranial pressure. Selection is performed, for example, to maintain and/or change (ICP) to be within a normal range and/or within a non-pathological range and/or to decrease ICP. Examples of maintained ICP ranges include: within 5-25 mmHg, or 7-15 mmHg; under 25 mmHg and/or 20 mmHg; above 5 mmHg; and/or another range of pressures (e.g., a range considered normal for a supine adult). For example, to maintain ICP and/or reduce ICP by no more than 1-30%, or 1-10%, or 5-30% or lower or higher or intermediate percentages or ranges. of a measured initial ICP before jetting and/or suction commences. In some embodiments, suction flow rate is 0.15-0.5 liters/minute, or 0.1-0.4 liters/minute, or 0.2-0.3 liters/minute, or lower or higher or intermediate ranges or flow rates.

In some embodiments, ICP is measured (e.g., by one or more sensors), assessed, and based on the assessment, a balance between fluid supply and suction is controlled to control the ICP. For example, sensed measurements are assessed to indicate that ICP is over a desired level, fluid supply rate and/or pressure is reduced and/or suction is increased.

In an exemplary embodiment, fluid flow supplied to the nozzle is about 0.2 liters/minute and suction flow rate is about 0.2-0.3 liters/minute.

In some embodiments, jetting is into an air (or gaseous) environment. For example, access channel(s) to the treatment site allow air flow to a region of the nozzle; e.g., in embodiments establishing and/or maintaining flow by suction at the tip. In some embodiments, characteristic(s) of the fluid jet (e.g., as defined by nozzle size and/or flow parameters) shape and/or are selected to provide gradual erosion, with the fluid being jetted in an air environment.

Alternatively, in some embodiments, a treatment area is maintained under fluid, and jetting is selected for gradual erosion of tissue when jetting in a fluid environment.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### System Example

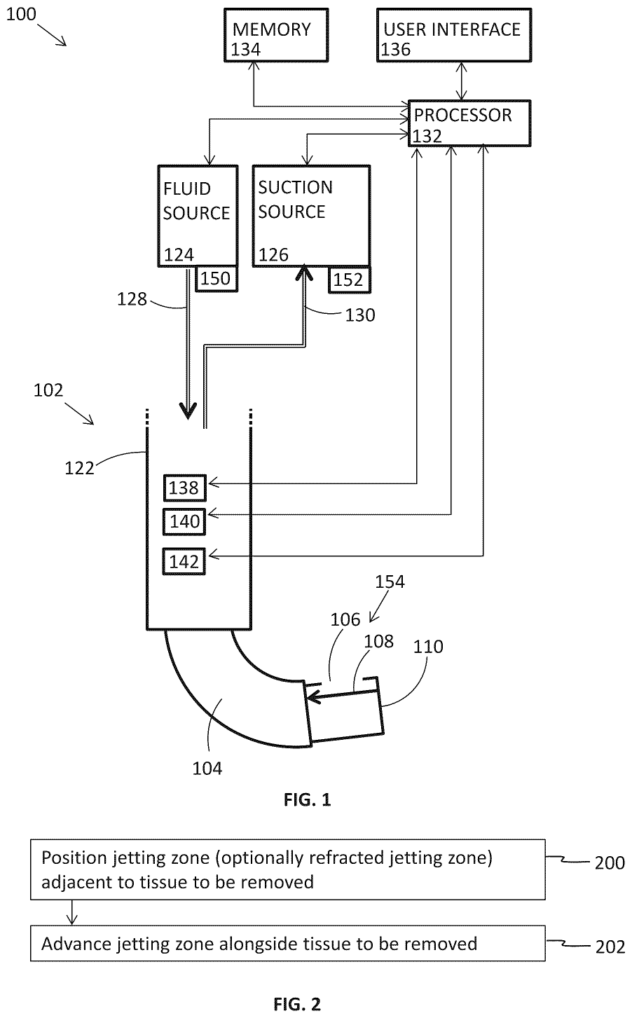

FIG. 1 is a simplified schematic of a system 100, according to some embodiments of the present disclosure.

In some embodiments, system 100 includes an arm 102.

In some embodiments, arm 102 includes a tip 154 disposed, in some embodiments, at a distal end of arm 102.

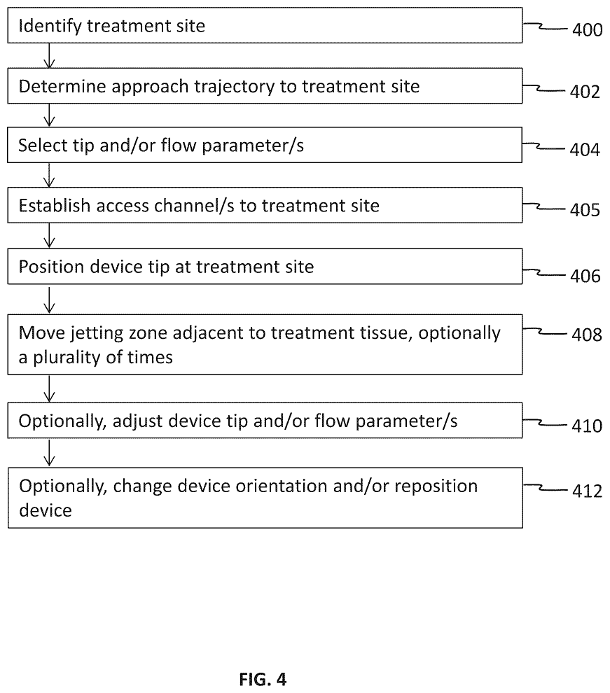

In some embodiments, arm 102 is configured to position tip 154 in a desired position in space, for example, in a desired position with respect to a treatment region of a subject. In some embodiments, arm 102 is configured to direct tip 154 and/or proximal portion(s) of arm 102 along a desired path; e.g., to arrive at a treatment region. Optionally the configuration is performed according to one or more features as described regarding step 405 and/or 406 of FIG. 4.

In some embodiments, arm 102 includes one or more of joint 104 and/or one or more of actuator 138. Optionally, actuator(s) 138 are configured to bend arm 102 at joint(s). In some embodiments, actuator(s) 138 are configured to advance one or more portions of arm 102.

In some embodiments, tip 154 is configured to provide a fluid flow 108 which, in some embodiments, interacts with tissue through one or more openings 106 in a body of tip 154 (opening 106, also herein termed “gap”). In some embodiments, fluid is jetted from arm 102 across opening 106 being refracted by a blocker 110 to provide fluid flow 108. Additionally or alternatively, in some embodiments, fluid supplied to tip 154 is refracted prior to exiting the tip into opening 106.

In some embodiments, arm 102 includes one or more arm sensors 140. In some embodiments, sensor(s) 140 sense one or more fluid parameters; for example fluid delivery parameter(s) (e.g., flow rate, temperature) and/or for example, extraction parameter(s) (e.g., suction pressure, flow rate). In some embodiments, sensor(s) 140 include one or more position sensors, for example, providing measurement(s) which are used to determine position of one or more portions of arm 102; e.g., with respect to external object(s) (e.g., patient tissue) and/or with respect to other portion(s) of the arm 102 and/or system 100.

Optionally, arm 102 and/or tip 154 includes one or more pressure sensors. For example, for monitoring pressure at a treatment site. Optionally, pressure is used as feedback for control of a tip fluid supply flow rate and/or a tip extraction rate and/or tip suction level.

Optionally, system 100 and/or arm 102 and/or tip 154 includes one or more optical sensors; e.g., a camera which, in some embodiments, is inserted along with tip 154. The camera, in some embodiments, providing visualization of a procedure to a professional carrying out the procedure. In some embodiments, a light sensor sensitive to fluorescent light is used to sense marker fluid present at a treatment site. Potentially assisting a user in identifying different tissue types at the treatment site.

Optionally, system 100 and/or arm 102 and/or tip 154 includes a light source; e.g., optical fiber, optionally to aid in visualization of the treatment site.

Optionally, system 100 and/or arm 102 and/or tip 154 includes a pressure sensor; e.g., providing feedback as to tissue characteristic(s).

Optionally, system 100 and/or arm 102 and/or tip 154 includes a temperature sensor.

In some embodiments, system 100 includes a fluid source 124. In some embodiments, system 100 includes one or more fluid source sensors 150, located, for example, at the fluid source 124; e.g., at an outlet to the fluid source. Sensors 150 are optionally used for control of parameter(s) of supplied fluid. In some embodiments, fluid source is connected to arm 102 by one or more fluid source conduits 128. In some embodiments, conduit(s) 128 extend through arm 102 to provide fluid for jetting 108 at tip 154.

In some embodiments, system 100 includes a suction source 126. In some embodiments, suction source 126 includes an extraction pump; e.g., a vacuum pump such as a peristaltic pump. In some embodiments, suction source 126 is connected to arm 102 by one or more suction conduits 130. Optionally, suction conduit(s) 130 extend through arm 102 to deliver suction to tip 154. Optionally, suction extracts jetted fluid and/or eroded tissue; e.g., through opening 106.

In some embodiments, system 100 includes a processor 132 which, in some embodiments, includes a controller which sends control signals to one or more of fluid source 128, suction source 136, and arm actuator(s) 138. In some embodiments, processor 132 receives sensor signals from one or more of arm sensor(s) 140 fluid source sensor(s) 150, and suction source sensor(s) 152. In some embodiments, sensor data and/or control data is stored and/or retrieved by processor 132 from a memory 134.

In some embodiments, system 100 includes one or more user interface 136 which receives user input(s) and/or displays information to a user.

In some embodiments, one or more of memory 134, user interface 136, and processor 132 are hosted locally to other system component(s) e.g., arm 102, for example, by circuitry attached and/or attachable to arm 102. Alternatively or additionally, one or more of memory 134, user interface 136, and processor 132 are at least partially hosted elsewhere; e.g., by the cloud, optionally using a user's portable electronic device (e.g., smartphone).

Optionally, in some embodiments, system 100 includes one or more additional tools; for example, one or more cameras (e.g., endoscope cameras). The cameras operate, for example, to provide visual feedback to a user of treatment; e.g., as it is carried out. For example, in some embodiments, arm 102 includes one or more cameras.

For example, in some embodiments, additional irrigation and/or suction is provided; e.g., by one or more additional conduits, the conduit(s) being optionally attached to and/or part of arm 102.

### Example Method of Tissue Removal

FIG. 2 is a method of tissue removal, according to some embodiments of the present disclosure.

At block 200, in some embodiments, a jetting zone is positioned adjacent to tissue to be removed. Optionally, in some embodiments, the jetting zone is a refracted jetting zone, and/or includes refracted jetted water.

At block 202, in some embodiments, the jetting zone is positioned adjacent to tissue to be removed. In some embodiments, the jetting zone is moved; e.g., back and forth, adjacent to and/or within tissue to be removed.

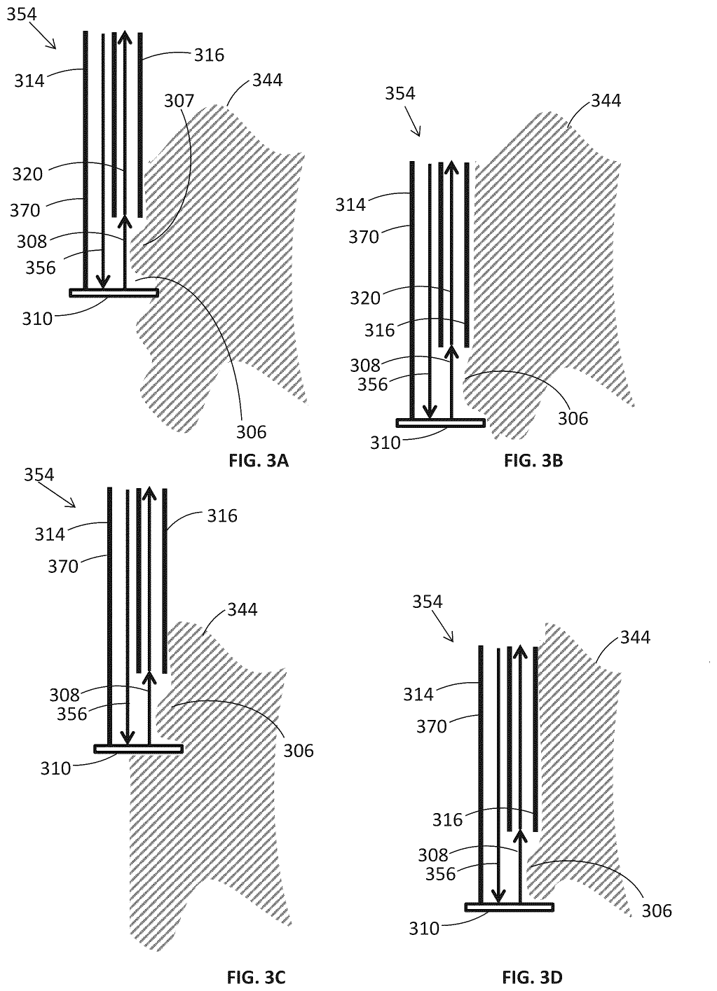

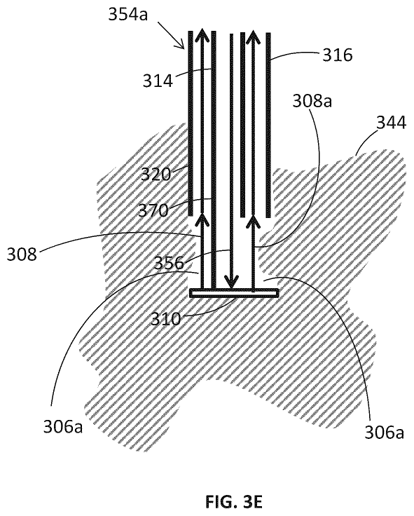

FIGS. 3A-3D are simplified schematics illustrating removal of target tissue 344, using a tip 354, according to some embodiments of the present disclosure.

In some embodiments, FIGS. 3A-3D sequentially illustrate progression of removal of target tissue 344, by positioning a jetting zone 308 adjacent to and/or moving jetting zone 308 past target tissue 344. In some embodiments, an opening 306 in a tip housing 370 to jetting zone 308 is positioned adjacent to tissue 344.

Jetting zone 308, in some embodiments, includes directly jetted fluid 356 and refracted jetted fluid 308, wherein the fluid is refracted by a blocker 310.

In some embodiments, tip 354 is pushed through tissue and/or between tissues. In some embodiments, tissue intrudes 307 into tip opening 306. For example, pressure of surrounding tissue pushes tissue 307 into opening 306; e.g., as tissue(s) are displaced by positioning (e.g., advancing and/or inserting) of the tip into tissue. For example, when removing tissue from the brain, pressure within the cranial cavity acts to push tissue into opening 306.

Alternatively or additionally, in some embodiments, tissue 307 intrudes into opening 306 under suction pressure applied through an extraction conduit 316. For example, were tissue to be removed is not under pressure.

FIGS. 3A-3D, in some embodiments, illustrate movement of tip 354 in two directions to remove target tissue 344. For example: for a plurality of directions (and/or starting positions) with respect to the target tissue, tip 354 is advanced and/or retracted. Advance and/or retraction is, for example, in a direction of elongation of the tip 354 and/or a distal portion of an arm to which the tip is attached.

FIG. 3E is a simplified schematic illustrating removal of target tissue 344, using a tip 354a, according to some embodiments of the present disclosure.

In some embodiments, target tissue 344 is removed by inserting tip 354a into the target tissue. Optionally, in some embodiments, target tissue 344 is eroded at more than one region, e.g., by a single tip and/or simultaneously. For example, as illustrated in FIG. 3E, tip 354 optionally includes first and second openings 306a (or a single opening which extends around the tip).

Optionally, in some embodiments, more than one water jet is provided; e.g., at least one jet per opening, for example as illustrated by refracted jets 308, 308a.

### Example of Method of Treatment

FIG. 4 is a flow chart of a method of treatment, according to some embodiments of the invention.

At block 400, in some embodiments, a treatment site is identified. For example, a medical professional selects a treatment site, e.g., a target tissue region for removal. Optionally, the medical professional selects the treatment site, using one or more types of measurement; e.g., imaging type(s).

In some embodiments, treatment sites are within the cranial cavity and/or include brain tissue.

In some embodiments, target tissue is cancerous and/or other diseased tissue targeted for removal. Alternatively or additionally, in some embodiments, selected target tissue includes healthy tissue. For example, in some embodiments, treatment includes removal of healthy tissue; e.g., healthy tissue surrounding and/or adjacent to diseased tissue, such as may be performed in the case of tremor and/or epilepsy treatment.

In some embodiments, a patient has more than one target region of tissue. Optionally, targets are separated from each other.

At block 402, in some embodiments, an approach trajectory to treatment site is determined. For example, for removal of tissue within the cranial cavity, the trajectory includes entrance point(s) to the cranial cavity and/or a path within the cranial cavity to the target region of tissue. In some embodiments, a path is determined for more than one tissue target. In some embodiments, a path includes movements of the tip during extraction of target tissue; e.g., including one or more movement features as described regarding FIGS. 3A-3D.

At block 404, in some embodiments, one or both of tip parameter(s) and flow parameter(s) are selected. For example, in some embodiments, a system (e.g., system 100) is compatible with a plurality of tips and/or arms; e.g., having different size and/or shape and/or jetting infrastructure and a tip(s) and/or flow parameter(s) are selected for particular treatment(s) and/or portion(s) of a treatment. In some embodiments, a number of tips to be used (e.g., concurrently and/or through different access channels) is selected. For each tip, in some embodiments, tip parameter(s) and/or flow parameters are selected.

Optionally, a first tip is selected for a first portion of a treatment, and additional tip(s) are then employed during later portion(s) of the treatment.

Optionally, at a beginning of a procedure, a coarse erosion set-up is employed; e.g., with one or more portions of the tip larger and/or more aggressive flow parameter(s) (e.g., higher flow and/or extraction rates). Using the coarse erosion set-up, a bulk of desired tissue is removed and/or a central region of tissue is removed and/or tissue to be removed not adjacent to healthy tissue is removed. In some embodiments, the tip and/or flow parameter(s) are then changed to a fine erosion setup, e.g., to remove remaining tissue to be removed. In the fine erosion setup, for example, one or more portions of the tip are larger, and/or flow parameters are less aggressive (e.g., lower flow and/or extraction rates).

At block 404, in some embodiments, access channel(s) to treatment site are established. For example, prior to positioning of the tip at the treatment site. Optionally, the tip itself is used to establish the channel. For example, in some embodiments, a tip includes a sharp and/or pointed portion for cutting through tissue.

At block 406, in some embodiments, the device tip is positioned at the treatment site; e.g., through the access channel. Optionally, the tip itself is used to establish the channel, allowing steps 404 and 406 to be performed as a single action.

At block 408, in some embodiments, the jetting zone is moved; e.g., adjacent to treatment tissue. Optionally a plurality of times, for example, for gradual removal of target tissue. In some embodiments, fluid and/or debris (e.g., from target tissue erosion) is extracted; for example, suctioned by an extraction conduit such as extraction conduit 316. In some embodiments, extraction is concurrent with erosion.

At block 410, optionally, in some embodiments, device tip and/or flow parameter(s) are adjusted. Optionally, adjustment is without withdrawing tip(s) from the treatment site.

In some embodiments, multiple tips are used. Optionally, an adjusted tip parameter is the number of tips at the treatment site, and/or which tips are used to remove tissue.

In some embodiments, a tip is withdrawn from the treatment site and a different tip is then positioned at the treatment site. Optionally, the same arm 102 is used for the different tips. The arm, in some embodiments, is compatible with more than one tip; e.g., is provided with a mount onto which a tip is removably attached.

At block 412, optionally orientation and/or position of a tip is changed. For example, the tip is repositioned and/or reoriented during erosion; e.g., to access regions of the target tissue. Optionally, a tip is withdrawn (e.g., at least partially) and re-inserted to a different position and/or at a different orientation with respect to the target tissue. Optionally, after withdrawal, the tip is changed and/or inserted through a different access channel and/or path.

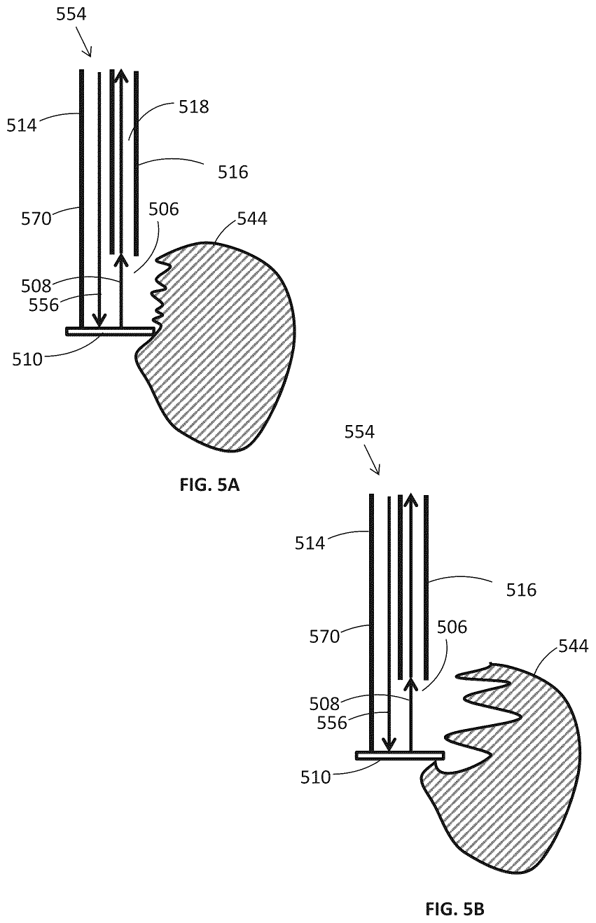

FIGS. 5A-5B are simplified schematics illustrating removal of target tissue 544, according to some embodiments of the present disclosure.

In some embodiments, a jetting zone is formed at tip 554 by jetted 556 and/or refracted jetted 508 fluid, refracted, e.g., by blocker 510. Interaction between the jetting zone and target tissue 544 is through an opening 506 in a housing 570 is configured to remove tissue. Fluid supply is through conduit 514. Extraction (via a flow of extracted fluid 518) is through conduit 516.

In some embodiments, the jetting zone and/or the tip opening are configured to gradually remove tissue, for example, as illustrated in FIG. 5A, and optionally not for erosion to extend away from the jetting zone; e.g., as illustrated in FIG. 5B.

Optionally, desired erosion is of pieces of tissue less than 0.1-2 mm³, or 0.1-1 mm³, or lower or higher or intermediate volumes or ranges.

In some embodiments, one or more jetting parameters (e.g., jetting pressure and/or jetting fluid flow) and/or one or more tip parameters (e.g., size of opening 506) is selected for gradual erosion of target tissue 544.

In some embodiments, different parameter(s) are selected for different treatment types and/or for different portions of a treatment. For example, in some embodiments, a stronger jet (e.g., higher pressure and/or flow rate) is selected for removal of tissue which is more resistant to erosion (e.g., denser and/or more fibrous).

### Examples of Tips

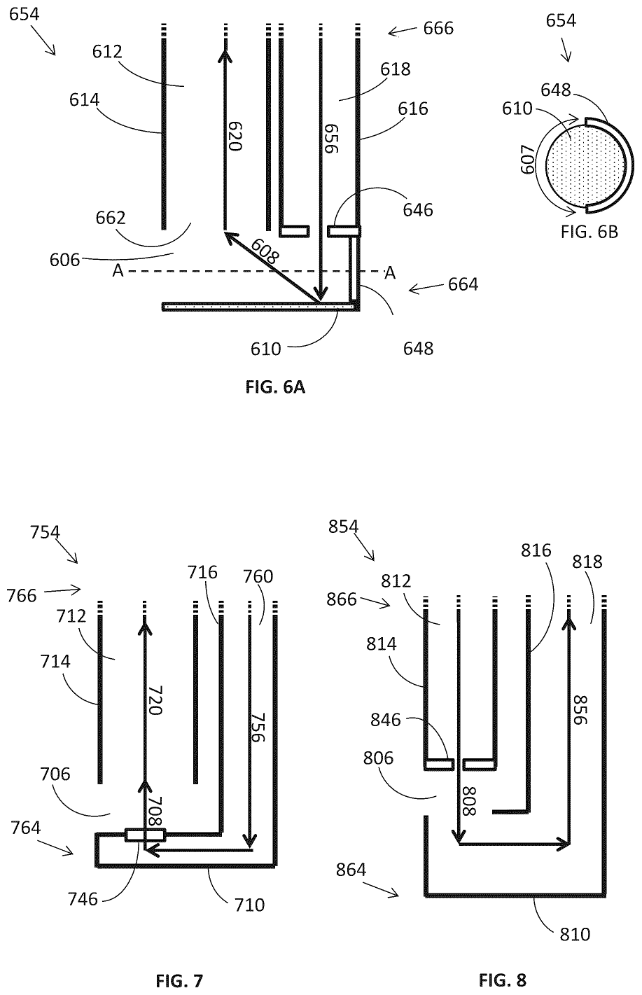

FIG. 6A is a simplified schematic cross section of a tip 654, according to some embodiments of the present disclosure.

FIG. 6B is a simplified schematic sectional view of a tip 654, according to some embodiments of the present disclosure. Distal side 664 and proximal side 666 are marked.

In some embodiments, tip 654 includes a supply conduit outlet 646, (which, in some embodiments, includes a nozzle 646) through which fluid passes across an opening 606. In some embodiments, incoming fluid 656 to nozzle 646 is supplied flowing through a supply lumen 618 of a supply conduit 614.

In some embodiments, after passing through nozzle 646 (e.g., being jetted from nozzle 646), fluid 608 is deflected (e.g., at least partially reflected) by a blocker 610. In some embodiments, blocker 610 blocks jetted fluid from extending distally of the blocker. In some embodiments, extracted fluid 620 is extracted through an extraction lumen inlet 662 of an extraction lumen 612 of an extraction conduit 616.

In some embodiments, conduits 614, 616 are coupled, for at least a portion of the conduit lengths. In some embodiments, coupling of conduits 614, 616 position portion(s) at desired position(s) e.g., to position supply outlet 646 and extraction inlet 662 in a desired spatial relationship to each other.

In some embodiments, supply conduit 614 has a supply conduit cross sectional area and extraction conduit 616 has an extraction conduit cross sectional area (cross sections taken, for example, transverse to a central long axis of the conduit and/or transverse to the proximal-distal direction). Optionally, the extraction conduit cross sectional area is larger than that of the supply conduit, for example, 1.2-10 times or 1.5-5 times, or lower or higher or intermediate multiples or ranges. A potential benefit of a wider extraction conduit is the ability to channel fluid and eroded tissue and/or other debris with reduced risk of blockage of the conduit.

In some embodiments, blocker 610 is connected to one or both of conduits 614, 616 by one or more connectors 648. In some embodiments, opening 606 is enclosed partially by connector 648. For example, referring to FIG. 6B which in some embodiments, illustrates a sectional view of tip 654 of FIG. 6A taken along line AA. Optionally, connector 648 defines a cross sectional circumferential extent 607 of opening 606. In some embodiments, connector 648 encloses 10%-80% of a cross sectional circumference of tip 654. Cross sectional circumferential extent 607 of opening 606 may form the remaining percentage of the cross sectional circumference of the tip.

FIG. 7 is a simplified schematic cross section of a tip 754, according to some embodiments of the present disclosure. Distal side 764 and proximal side 766 are marked.

In some embodiments, fluid 756 supplied through a supply lumen 760 of a supply conduit 716 is passed across an opening 706 in tip 754 as jet 708, in a distal-proximal direction. In this embodiment, blocker 710 simply functions as a portion of supply conduit 716.

In some embodiments, a supply conduit outlet is provided by a nozzle 746 for jetting of fluid 708 across opening 706.

In some embodiments, extracted fluid 720 is extracted through an extraction lumen inlet of an extraction lumen 712 of an extraction conduit 714.

FIG. 8 is a simplified schematic cross section of a tip 854, according to some embodiments of the present disclosure. Distal side 864 and proximal side 866 are marked.

In some embodiments, fluid supplied through a supply lumen 812 of a supply conduit 814 is passed (after leaving nozzle aperture 846) across an opening 806 in tip 854 in a proximal-distal direction. Fluid and/or debris is removed through an extraction lumen 818 of extraction conduit 816, which reverses direction of low from proximal-distal to distal-proximal.

In some embodiments, the extraction conduit 816 itself provides a blocker 810 to passage of supplied (e.g., jetted) fluid 808. In some embodiments, cross sectional (e.g., taken transverse to fluid flow) area of extraction lumen 818 is larger than that of supply lumen 812; dimensions, for example, as described regarding cross sectional area of extraction conduit 616 of FIG. 6A.

In some embodiments, an extraction conduit forming blocker 810, and/or an extraction conduit passing across a distal end of tip 854 positions opening 806 further proximal than, for example, the opening position in other tip configurations; e.g., that of FIG. 6A and/or FIG. 7.

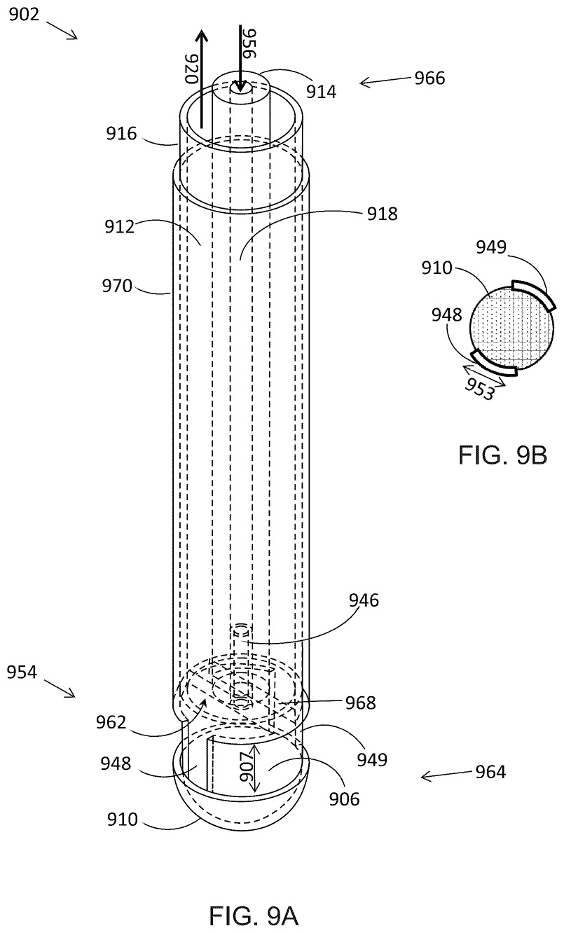

FIG. 9A is a simplified schematic of a portion of an arm 902 including a tip 954, according to some embodiments of the present disclosure.

FIG. 9B is a simplified schematic sectional view of a tip 954, according to some embodiments of the present disclosure.

In some embodiments, arm 902 has a distal end 964 and extends proximally 966; e.g., towards fluid and/or suction supplies (not illustrated).

In some embodiments, a supply conduit 916 supplies fluid to tip 954. Optionally, supply conduit 916 is fluidly connected to a nozzle 946 through which fluid is jetted across an opening 906. Optionally, opening 906 is a space through which tissue interacts with jetted fluid. In some embodiments, opening 906 is between nozzle 946 and a blocker 910. Blocker 910, in some embodiments, prevents flow of fluid distal of the blocker.

In some embodiments, suction supplied through an extraction lumen 912 of an extraction conduit 916 extracts fluid and/or debris through an extraction lumen inlet 962 of extraction conduit 916.

In some embodiments, supply conduit 916 is disposed within extraction conduit 916; optionally extending through extraction conduit 916. In some embodiments, supply conduit 916 is disposed in a central region of extraction conduit, extraction lumen 912 in some embodiments, having an annular cross section in a cross section taken in a transverse direction to a proximal-distal direction with respect to arm 902 and/or tip 954.

In some embodiments, nozzle 946 is fluidly connected to supply conduit 914. For example, by inserting of a nozzle component 946 into supply conduit 916; e.g., into a distal end of supply conduit 914.

Optionally, in some embodiments, nozzle component 946 is adhered to supply conduit 916; e.g., by adhesive. Alternatively or additionally, in some embodiments, supply conduit 916 is elastic and nozzle component 946 has a larger cross section than supply conduit 916 cross section when elastically relaxed, the supply conduit 916 stretching to accommodate nozzle component 946 and elastic relaxation force of the conduit acting to hold the nozzle component 946 in position.

In some embodiments, supply conduit 916 includes metal (e.g., is reinforced with metal optionally as a braid or other metal structure). In some embodiments, nozzle component includes metal; e.g., is formed of a metal such as stainless steel. In some embodiments, supply conduit 916 and nozzle component 946 are welded together.

In some embodiments, supply conduit 916 and/or nozzle 946 are held in position, for example, with respect to extraction conduit 916 and/or extraction lumen 912. For example, to prevent supply conduit 916 and/or nozzle 946 from extending distally and/or retracting from extraction conduit 916; e.g., with movement of arm 902. In some embodiments, a holder 968 holds nozzle 946 and/or supply conduit 916 in position, for example, distal ends of nozzle 946 and/or supply conduit 914. In some embodiments, holder 968 is connected to extraction conduit 916, e.g., at one or more regions of extraction conduit 916. In an exemplary embodiment, holder 968 bridges extraction conduit 916, being attached to two parts of extraction conduit 916 and bridging a space between the parts. In some embodiments, holder 968 passes across a cross sectional center of extraction conduit 916. In an exemplary embodiment, the holder 968 holds the nozzle and/or supply conduit at a cross sectional center of extraction conduit 916.

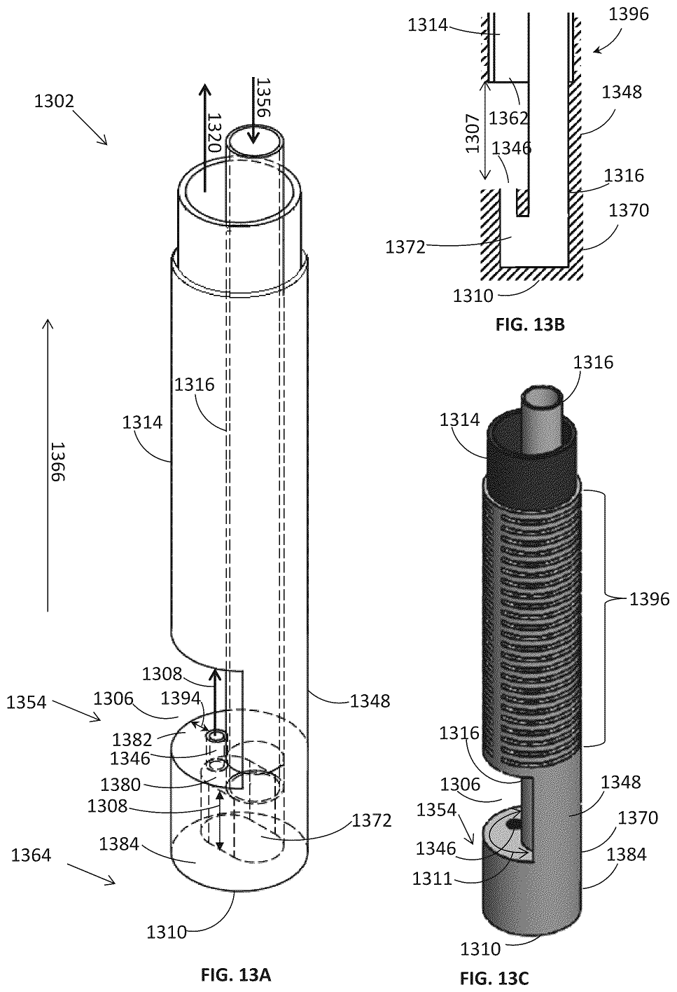

In some embodiments, arm 902 includes an outer sheath 970. In some embodiments, bending of supply and/or extraction conduits 914, 916 is effected by bending of outer sheath 970; e.g., including one or more features as illustrated regarding outer sheath 1370 of FIGS. 13A-13C. In some embodiments, blocker 910 is connected to outer sheath 970; e.g., at a distal end of outer sheath 970. In some embodiments, connection is by one or more connectors 948, 949. Optionally, un-connected portions of sheath 970 and/or blocker 910 form opening 906 through which fluid interacts with tissue.

FIGS. 9A and 9B illustrate an exemplary embodiment including two connectors dividing opening 906 into two opposing portions. Optionally, the connectors have a same cross section extending distally from sheath 970 to blocker 910. In some embodiments, one, or more than two, or 2-8, or lower or higher or intermediate numbers of connectors connect blocker 910 to proximal portion(s) of arm 902 and/or tip 954. Optionally, additional connectors divide opening 906 further; for example, connectors are spaced with portions of opening 906 therebetween.

In some embodiments, one or more connectors has a differing cross sectional extent in one or more direction moving in a proximal-distal direction. In some embodiments, connector(s) are sized and/or shaped to provide desired dimension and/or shape opening(s).

In some embodiments, blocker 910 extends transversely to a direction of elongation of arm 902 to cover an entirety of extraction inlet 962 and/or a volume defined by outer sheath 970 and/or a volume defined by extraction conduit 916. A potential benefit of such a transverse extent to the blocker is that if maximally blocks jetted fluid from impinging onto tissue whilst maintaining a minimal transverse dimension of the arm (potentially enabling minimally invasive positioning of the arm and/or tip for treatment).

In some embodiments, blocker 910 includes a rounded and/or blunt shaped external distal portion. In some embodiments, blocker 910 has a hemispherical outer surface. In some embodiments, blocker 910 has walls which extend proximally; e.g., curving proximally from blocker 910 distal end. In some embodiments, a blocker has a distal end portion and walls which extend proximally of the distal end portion. For example, in some embodiments, a hemispherical shaped distal end portion has cylindrical walls extending proximally from edges of the hemisphere. In some embodiments, the inner walls of blocker 910 are concave; e.g., have a dome (hemispherical) shape. The concavity, e.g., in contrast to a planar blocker, potentially refracting jetted water into a narrower beam. In some embodiments, a shape of the walls (e.g., inner surface of the blocker walls) is selected to produce desired refracted fluid characteristics.

In some embodiments, radius of curvature of one or more portions of a blocker inner and/or outer surface are 1-20 mm, or 2-20 mm, or 2-10 mm, or lower or higher or intermediate radii or ranges.

In some embodiments, blocker 910 has a concave inner surface. In some embodiments, the shape of the blocker inner surface is selected to minimally broaden the incident beam of fluid jetted through nozzle 946 upon impinging on the blocker surface.

In some embodiments, an inner surface of blocker 910 extends to form a point (e.g., has a conical shape). For example, slopes of the inner surface increase at a central region of the inner surface, and/or increase at a region where the water jet impinges on the surface. A potential benefit of this is focusing of the refracted jetted water beam.

In some embodiments, a region where the water jet impinges on the inner surface includes a protrusion which potentially guides the water jet; e.g., through surface tension. A potential benefit being focusing of the refracted jetted water beam.

Optionally, in some embodiments, one or more portions of tip 954 are adjustable. For example, a size and/or circumferential position of gaps 906. For example, in some embodiments, outer sheath 970 is extendable and/or retractable to increase and/or decrease respectively a length (height) 907 of gap 906. For example, in some embodiments, outer sheath 970 and/or connector 948 are rotatable with respect to inner portion(s) of arm 902. Optionally, rotation of the outer sheath 970 and/or connector 948 (e.g., around a central longitudinal axis of arm 902 and/or tip 954) moves a position of connector 948 and/or gap 906; e.g., for changing region(s) of tissue eroded at the jet-tissue interface provided by gap 906.

In some embodiments, a maximal cross sectional extent (e.g., diameter for cylindrical portions) of tip 954 and/or of arm 902, where the cross section is taken transverse to a proximal-distal direction is 0.5-10 mm, or 0.5-5 mm, or 1-5 mm, or 2-3 mm, or lower or higher or intermediate ranges or distances.

In some embodiments, a height 907 of gap 906 is 0.5-4 mm, or 0.5-3 mm, or 0.5-2.5 mm, or 1-3 mm, or 1-2 mm, or 2-3 mm, or about 2.5 mm or lower or higher or intermediate heights or ranges.

In some embodiments, a width of gap 906 is 0.5-8 mm, or 0.5-5 mm, or 1-5 mm, or 3-6 mm, or lower or higher or intermediate widths or ranges. In some embodiments, the width of gap 906 is measured of a largest plane position-able in the gap and/or is measured along a circumference of the gap as described by portion(s) distal to the gap (e.g., of blocker 910) and/or by portion(s) proximal of the gap; e.g., outermost portion 970 of the tip proximal of the gap.

In some embodiments, height 907 and width 906 of gap have about the same dimensions and/or are within 5%, or 10%, or 20%, or 50%, of each other.

Referring now to FIG. 9B: in some embodiments, a central long axis length 953 of one or both of connectors 948, 949 is 0.1-5 mm, or 0.1-2 mm, or about 0.5 mm, or lower or higher or intermediate lengths or ranges.

Referring now back to FIG. 9A: in some embodiments, a maximal cross sectional extent (e.g., diameter for cylindrical portions) of supply conduit 914, where the cross section is taken transverse to a proximal-distal direction is 0.5-3 mm, or 1-2 mm, or about 1.6 mm. Optionally, a wall thickness of supply conduit 956 is 0.01-0.5 mm, or 0.05-0.3 mm, or 0.1-0.3 mm, or about 0.2 mm, or lower or higher or intermediate ranges or thicknesses.

In some embodiments, a maximal cross sectional extent (e.g., diameter for cylindrical portions) of extraction conduit 916, where the cross section is taken transverse to a proximal-distal direction is 0.5-5 mm, or 1-5 mm, or 2-4 mm, or about 3 mm. Optionally, a wall thickness of supply conduit 956 is 0.05-1 mm, or 0.1-0.5 mm, or 0.2-0.4 mm, or about 0.3 mm, or lower or higher or intermediate ranges or thicknesses.

In some embodiments, one or more of extraction conduit 916 (through which extraction fluid 920 flows) and supply conduit 914 (defining supply lumen 918) are sufficiently mechanically strong to resist rupture and/or collapse under pressure of fluid flow therewithin. In some embodiments, one or more of extraction conduit 916 and supply conduit 914 include a scaling material (e.g., polymer) which is, in some embodiments, reinforced, for example, with metal; e.g., steel and/or nitinol. Reinforcing is, in some embodiments, by braid and/or mesh and/or a cut tube.

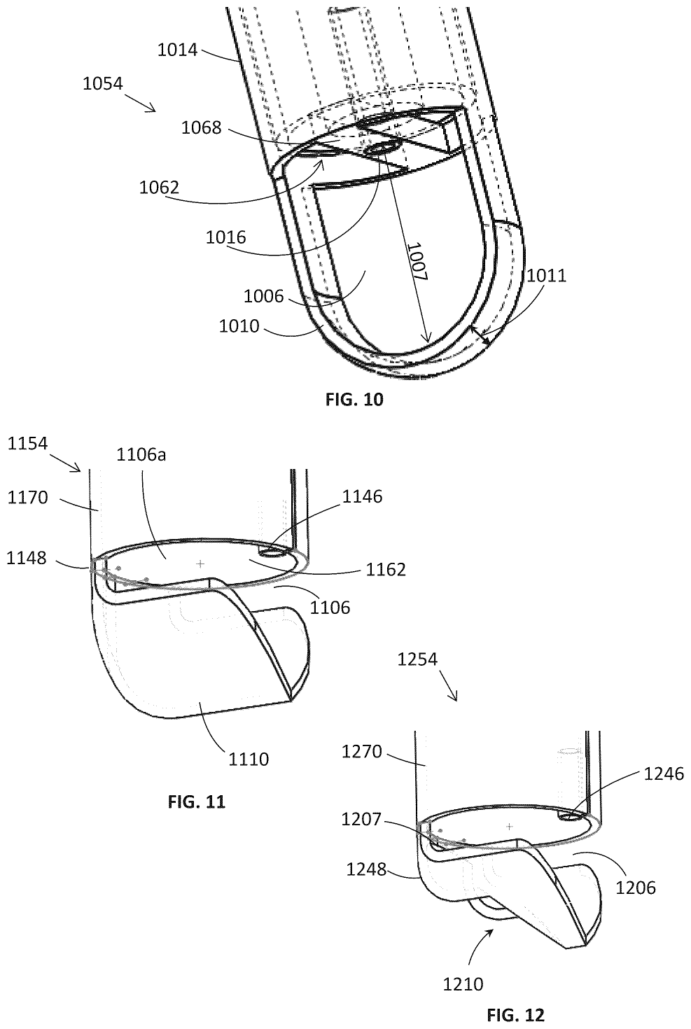

Reference is now made to FIG. 10, which illustrates a simplified schematic of a tip 1054, according to some embodiments of the present disclosure.

In some embodiments, tip 1054 includes a supply conduit 1016 which, in some embodiments, includes one or more properties of supply conduit 914 of FIG. 9A. In some embodiments, tip 1054 includes a holder 1068 which, in some embodiments, includes one or more properties of holder 968 of FIG. 9A. In some embodiments, tip 1054 includes an extraction conduit 1014 which, in some embodiments, includes one or more properties of extraction conduit 916 of FIG. 9A.

In some embodiments, a blocker 1010 blocks a portion of a cross section of extraction conduit 1014 and/or extraction inlet 1062. Optionally, a central region of jetted fluid is blocked. Optionally, edge portion(s) of the fluid are allowed to continue in a direction of jetting; e.g., to interact with tissue distal of tip 1054. In some embodiments, a majority of jetted fluid is refracted proximally and/or to the sides by interaction with blocker 1010. For example, a width 1011 of blocker being larger than a cross sectional diameter of jetted fluid impinging on blocker 1010. In some embodiments, at least a portion of fluid flows along a surface of blocker 1010. Blocker 1010, in some embodiments, forming a water flow aided cutting surface.

In some embodiments, blocker 1010 has a curved shape; e.g., extending between sides of tubular structure 1014.

In some embodiments, one or both of holder 1068 and blocker 1010 are attached to extraction conduit 1014. Tip 1054 optionally, in some embodiments, not including an outer sheath. Alternatively, or additionally, in some embodiments, one or both of holder 1068 and blocker 1010 are attached to an outer sheath 1014 which includes, in some embodiments, one or more properties of outer sheath 970 of FIG. 9A. Optionally, FIG. 10 does not illustrate an extraction conduit.

In some embodiments, tip 1054 does not include a separate connector for connection of blocker 1010 to proximal portion(s) of tip 1054, blocker 1010, in some embodiments, being directly attached to extraction conduit 1014 and/or outer sheath.

In some embodiments, blocker 1010 has a width 1011 of 0.5-5 mm, or 0.5-2 mm, or lower or higher or intermediate widths or ranges. In some embodiments, a height 1007 of a gap 1006 between an outlet to supply conduit 1016 and a distalmost portion of blocker 1010.

Reference is now made to FIG. 11, which is a simplified schematic of a tip 1154, according to some embodiments of the present disclosure.

In some embodiments, tip 1154 includes a blocker 1110 which extends transversely to a direction of elongation of an outer sheath and/or extraction conduit to cover an entirety of an extraction inlet 1162. Optionally, e.g., as described with regards to FIG. 10, element 1170 is an outer sheath and/or extraction conduit (e.g., as described elsewhere in this document).

In some embodiments, blocker 110 and/or a connector 1148 connecting blocker 1110 to element 1170 are shaped to provide an opening 1106 having different dimensions at different sides of tip 1154.

For example, in some embodiments, a region of the circumference gap 1106 extends from inlet 1162 to a distal end of the tip (e.g., less a thickness of blocker 1110). Optionally, blocker 1110 edges curve proximally for some but not all regions of the blocker. In an exemplary embodiment illustrated in FIG. 11, a single region of the gap is wider than other regions of the gap 1106 and the tip includes a single connector 1148 (which fully encloses a portion of a circumference of jetted and/or refracted fluid). Optionally, the wider portion of the gap and the connector are at opposing sides of the tip. In some embodiments, a gap 1106 between supply conduit outlet 1146 and blocker 1110 has different dimensions at different regions of a circumference of tip (e.g., cross sectional circumference of element 1170). In some embodiments, gap 1106 includes a portion sized and/or shaped to interface with (e.g., receive) tissue to be eroded. In some embodiments, other circumferential regions do not include a gap. For example, referring to FIG. 11: in some embodiments, a narrower portion of gap 1106a does not exist. Rather, connector 1148 extends circumferentially around the tip except for at the area of gap 1106 itself. In some embodiments, tip 1154 with gap portion 1106a (e.g., as illustrated in FIG. 11) is potentially easier to manufacture than a tip having only gap 1106. For example, connection between element 1170 and connector 1148 has a smaller extent, which potentially requires less gluing and/or welding.

In some embodiments, an outlet 1146 of a supply conduit is located non-centrally with respect to one or more of blocker 1110 and element 1170. In some embodiments, outlet 1146 is adjacent to a portion of opening 1106; e.g., to the wider portion of opening 1106.

Reference is now made to FIG. 12, which is a simplified schematic of a tip 1254, according to some embodiments of the present disclosure.

In some embodiments, tip 1254 includes a blocker 1210 having a sharp angled edge and/or tip. A potential benefit being the ability to cleave and/or cut tissue during positioning of the tip and/or in selecting and/or preparing tissue to be eroded.

In some embodiments, tip 1254 has one or more features as illustrated and/or described regarding tip 1154 of FIG. 11. For example, tip 1254 having a gap 1206 having different dimensions around a cross sectional circumference of the tip. For example, positioning of supply outlet 1246 with respect to element 1270. Element 1270 is an outer sheath and/or extraction conduit (e.g., as described elsewhere in this document). For example, in some embodiments, tip 1254 has a connector 1248 (connector 1248 including one or more features of connector 1148). In some embodiments, connector 1248 includes one or more of hole 1207. Hole 1207, in some embodiments, increasing case of manufacture.

FIGS. 13A-13C present simplified schematic views of a portion of an arm 1302 including a tip 1354, according to some embodiments of the present disclosure.

Referring now to FIG. 13A: in some embodiments, tip 1354 is disposed at a distal end 1364 of arm 1302. Arm 1302 extends in a distal-proximal direction 1366 from tip 1354.

In some embodiments, arm 1302 includes a supply conduit 1316 for supply of fluid 1356 to tip 1354 and an extraction conduit 1314 for extraction (e.g., by suction) of fluid 1320 and/or debris.

In some embodiments, fluid 1308 is jetted across an opening 1306 in tip 1354 proximally 1366 (or a direction including a proximal component). For example, fluid supplied through a supply conduit 1316 is refracted; e.g., prior to passing into opening 1306 (e.g., being jetted through a nozzle 1346).

In some embodiments, opening 1306 has a height 1307 and a width 1311. Optionally, height 1307 is measured as a shortest distance between the outlet of nozzle 1346 and an inlet 1362 of extraction conduit 1314. In some embodiments, width 1311 is measured as a portion of a circumference of the opening in outer sheath 1370. Alternatively or additionally, in some embodiments, a width of opening 1306 is measured as a largest dimension of a volume of opening 1306 perpendicular to distal-proximal direction 1366.

Optionally, the opening height and/or width is 0.5-10 mm, or 1-5 mm, or lower or higher or intermediate ranges or values.

In some embodiments, fluid passes from supply conduit 1316 into a reservoir 1372 which has a larger lateral extent than supply conduit 1316; e.g., extending laterally from supply conduit, at least a portion of reservoir 1372 overlapping with a distal side of opening 1306. Reservoir 1372, in some embodiments, having a portion 1380 which extends away from supply conduit 1316 in one or more directions. In some embodiments, nozzle 1346 is fluidly connected to reservoir 1372 at extending portion 1380.

In some embodiments, reservoir 1372 is defined by a base 1310, a cover 1382 (optionally including an outlet for nozzle 1346), and walls 1384. In some embodiments, walls 1384 are formed by a portion of outer sheath 1370.

In some embodiments, portions of tip 1354, distal of opening 1306 (e.g., one or more of nozzle 1346, reservoir 1372, base 1310, reservoir walls 1384) are connected to portion(s) proximal of 1306 by one or more connectors 1348. In some embodiments, a portion of outer sheath 1370 forms connector 1348.

In some embodiments, supply conduit 1316 is not centrally disposed centrally within extraction conduit 1314. For example, in some embodiments, being disposed so that together the outlet of nozzle 1346 and supply conduit 1316 occupy a central region of extraction conduit 1314 and/or arm 1302.

In some embodiments, a position of the outlet of nozzle 1346 is selected to be at a cross sectional central region of arm 1302 and/or at a separation 1394 from edges and/or an outer contour of arm 1302; e.g., as defined by outer sheath 1370. Selection optionally places a tissue-jetted fluid 1308 interface is at a distance from a central axis of jetted fluid 1308. A potential benefit of this is gradual tissue erosion.

Referring now to FIG. 13B and FIG. 13C: in some embodiments, tip 1354 is positioned by bending of arm 1302 at one or more joints. Optionally, position of tip 1354 is controlled by control of bending of a joint 1396 provided by a flexible portion of outer sheath 1370. In some embodiments, other portion(s) of outer sheath 1370 include rigid material; e.g., the portion of outer sheath 1370 at tip 1354. In an exemplary embodiment, outer sheath 1370 is formed from a rigid tubular structure (e.g., a stainless steel tube) which is then cut (e.g., laser cut) to provide flexible portion 1396.

In some embodiments, one or more of conduits 1314, 1316 are formed from a rigid structure which is cut to provide flexible portions. Optionally, at least cut portions are covered and/or connected to provide fluid scaling.

In some embodiments, one or more of conduits 1314, 1316, and outer sheath 1370 are formed from a first material reinforced by a second material; for example, reinforced by a plastic and/or polymer, and/or reinforced by metal (e.g., metal braid, mesh, and/or helix). Optionally, the first material provides sealing to the tubular structure and where, in some embodiments, the second material provides mechanical strength; e.g., sufficient to resist bursting and/or collapse under pressures of flows therewithin.

In some embodiments, a distal portion of tip 1354; e.g., housing the nozzle and/or reservoir, is rigid; e.g., is formed of a rigid material such as steel.

In some embodiments, one or both of supply conduit 1316 and extraction conduit 1314 are flexible, at least at portions of the conduits aligned with flexible portion 1396.

In some embodiments, supply conduit 1316 and extraction conduit 1314 are separate elements which are coupled (e.g., by insertion of supply conduit 1316 into the lumen of extraction conduit 1314). In some embodiments, supply conduit 1316 and extraction conduit 1314 are attached, for example, adhered to each other and/or formed as a single element.

In some embodiments, extraction conduit 1316 and outer sheath 1370 are connected to form a single structure. For example, the two elements being supplied by a single structure where mechanical properties are provided by outer sheath 1370, but having additional layer(s) which provide fluid enclosure to and/or sealing of flexible portion 1396.

Referring now back to FIG. 13A: in some embodiments, reservoir 1372 is bounded, at least partially (e.g., at least part of cover 1382 and/or walls 1384 and/or base 1310) by portions sufficiently rigid to prevent significant deformation of the reservoir; e.g., potentially preventing restriction of supply of fluid to nozzle 1346.

In some embodiments, blocker 1310 (e.g., blocker distal-most surface) includes one or more rounded and/or sharp portions to assist in positioning of tip and/or manipulating tissue with the tip.

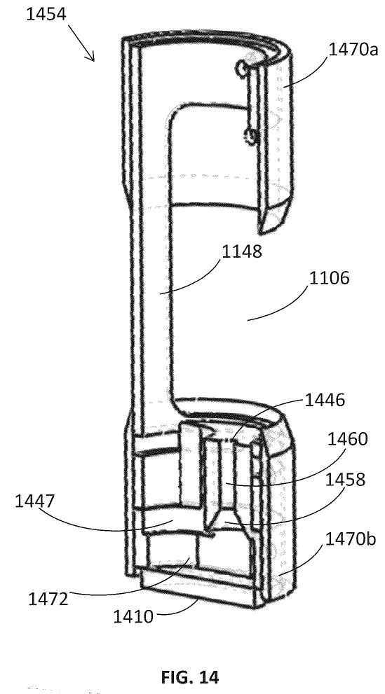

FIG. 14 is a simplified schematic section view of a tip 1454, according to some embodiments of the present disclosure.

In some embodiments, FIG. 14 illustrates a section view of tip 1454 where the section is taken in a direction parallel to a direction of elongation of tip 1454 and/or an arm to which tip 1454 forms a distal portion.

In some embodiments, FIG. 14 illustrates a sectional view of tip 1354 of FIG. 13A.

In some embodiments, tip includes an outer sheath 1470a, 1470b which, in some embodiments, is a tubular structure, in which a hole is cut to provide a gap 1106.

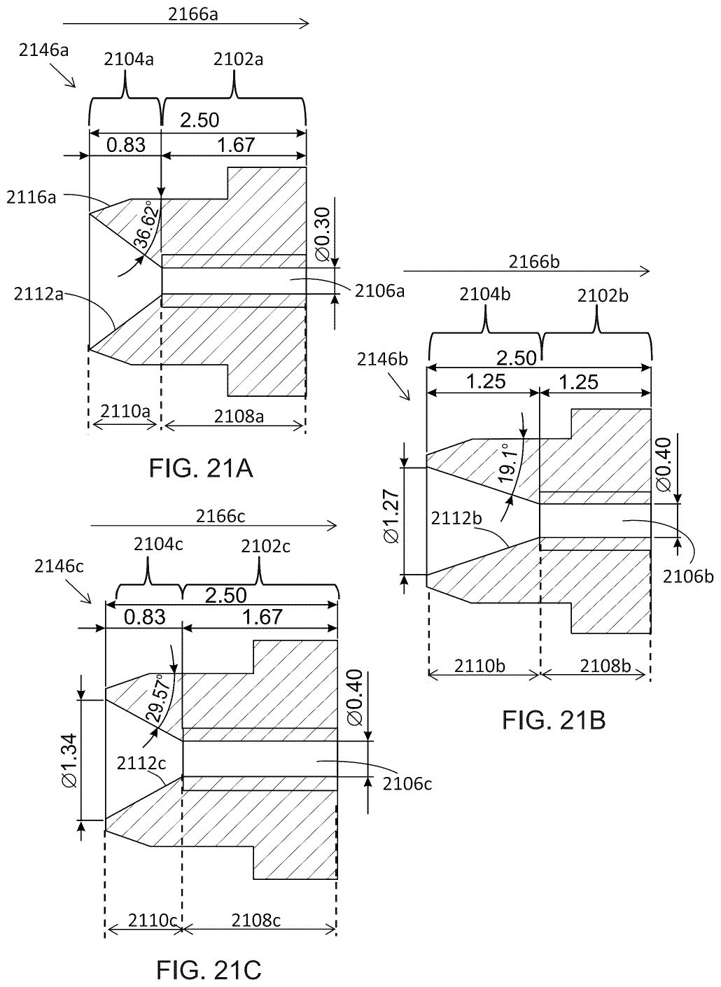

In some embodiments, base 1410 is attached to a distal end of outer sheath 1470b. In some embodiments, a nozzle portion 1447 defines, between base 1410 and gap 1106, a fluid supply outlet 1446 and a reservoir 1472. In some embodiments, fluid delivered to reservoir 1472 (e.g., through a supply conduit not illustrated in FIG. 14) flows through a narrowing 1458 before passing through nozzle tubing 1460 leading to outlet 1446. In some embodiments, narrowing 1458 has angled walls, providing a truncated cone-shape. Exemplary embodiments for narrowing 1458 and/or nozzle tubing 1460 are illustrated in and/or described regarding FIGS. 21A-21C.

In some embodiments, tip 1454 includes a connector portion 1148 extending from a connection with element 1470a proximal of gap 1106 to a connection with element 1470b distal of gap 1106. In some embodiments, connector portion 1148 acts to reinforce element 1470a, 1470b and/or connection between portions of the tip proximal and distal of gap 1106.

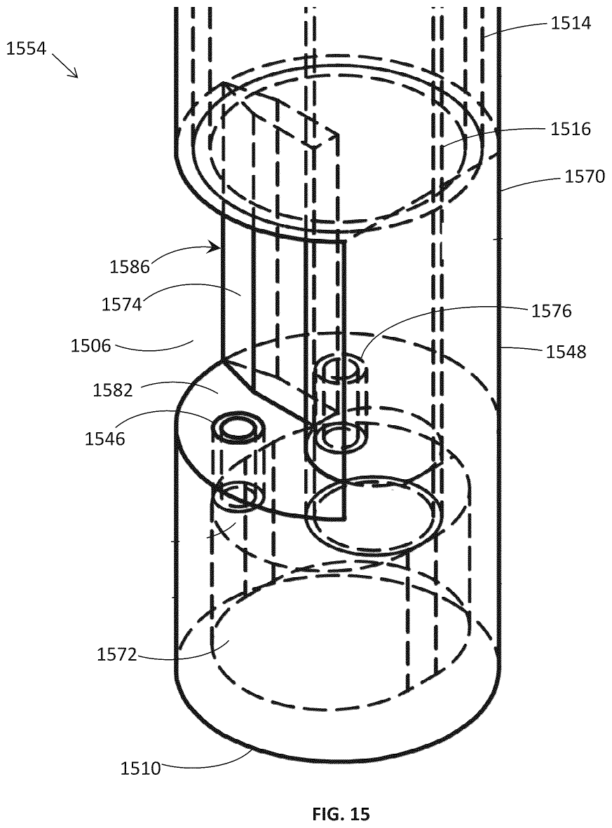

FIG. 15 is a simplified schematic of a tip 1554, according to some embodiments of the present disclosure.

In some embodiments, tip 1554 includes one or more features as illustrated in and/or described regarding tip 1354 in FIGS. 13A-13C. Optionally, tip 1554 includes a supply conduit 1516, an extraction conduit 1514, a reservoir 1572 (for which blocker 1510 provides a distal end cap) and, optionally, an outer sheath 1570 including feature(s) of one or more of: supply conduit 1316, extraction conduit 1314, reservoir 1372 and outer sheath 1370, respectively, of FIGS. 13A-13C.

In some embodiments, fluid is supplied to an opening 1506 through a plurality of outlets 1546, 1576. Optionally, outlets are embodied as nozzles 1546, 1576.

In some embodiments, tip 1554 includes one or more dividers 1574 disposed within opening 1506. In some embodiments, divider 1574 divides opening 1506, for example, in a proximal-distal direction. In some embodiments, divider 1574 acts to cleave tissue pressing against the tip; divider 1574 including, in some embodiments, a tapered and/or sharp edge 1586. Optionally, edge 1586 is orientated facing outwards from divider 1574. In some embodiments, divider 1574 extends to an outer surface (or within 0.5 mm) of an outer contour of tip 1554 and/or opening 1506; e.g., as defined by sheath 1570 and/or edges of reservoir top 1582. In some embodiments, divider 1574 extends outwards from a body of tip 1554; for example, divider 1574 extends outwards from outer sheath 1570 and/or edges of reservoir top 1582 by 0.1-1 mm.

In some embodiments, divider 1574 acts as a connector between portions of tip 1554 distal of opening 1506. In some embodiments, additionally or alternatively to divider 1574, a connector 1548 connects portion(s) proximal of opening 1506 and those distal of opening 1506. In some embodiments, connector 1548 is a portion of outer sheath 1570.

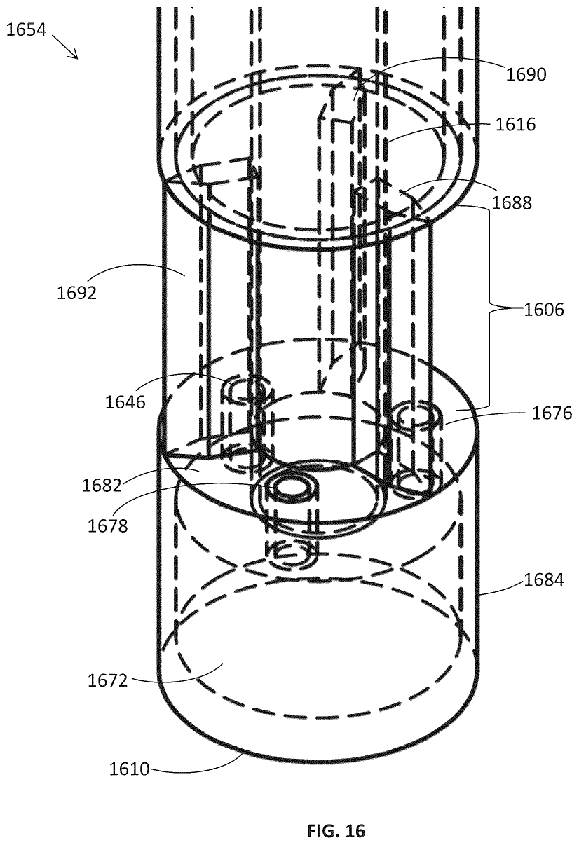

FIG. 16 is a simplified schematic of a tip 1654, according to some embodiments of the present disclosure.

In some embodiments, tip 1654 includes one or more features as illustrated for and/or described regarding tip 1554 of FIG. 15.

In some embodiments, tip 1654 includes a plurality of nozzles 1646, 1676, 1678. In some embodiments, nozzles 1646, 1676, 1678 provide outlets of a reservoir 1672 fluidly connected to a supply conduit 1616. Alternatively, in some embodiments, each nozzle 1646, 1676, 1678 has its own reservoir and/or supply conduit. In some embodiments, nozzles 1646, 1676, 1678 are located at different positions of a cross section of the nozzle tip taken transverse to a direction of elongation of the tip and/or arm. In some embodiments, for example as illustrated in FIG. 16, nozzles 1646, 1676, 1678 are located in a same proximal-distal position. Alternatively, in some embodiments, a tip includes a plurality of nozzles distributed at different proximal-distal positions and/or different transverse positions.

A potential benefit of a tip having a plurality of jetting zones (e.g., associated with a plurality of nozzles 1646, 1676, 1678) is the ability to erode tissue in different directions around tip 1654, optionally without moving and/or rotating tip 1654.

In some embodiments, tip includes a plurality of dividers 1688, 1690, 1692 which, in some embodiments, divide between jetting zones of the nozzles. A jetting zone is defined as a portion of an opening 1606 inhabited by fluid jetted from an associated nozzle.

In some embodiments, divider(s) 1688, 1690, 1692 provide connection between portion(s) of tip 1654 distal of opening 1606 (e.g., including one or more of reservoir 1672, nozzles 1646, 1676, 1678, reservoir base 1610, reservoir top 1682, reservoir walls 1684) and portion(s) proximal of opening 1606.

In some embodiments, divider(s) 1688, 1690, 1692 additionally or alternatively provide a cutting edge; e.g., as described regarding divider 1586 of FIG. 15.

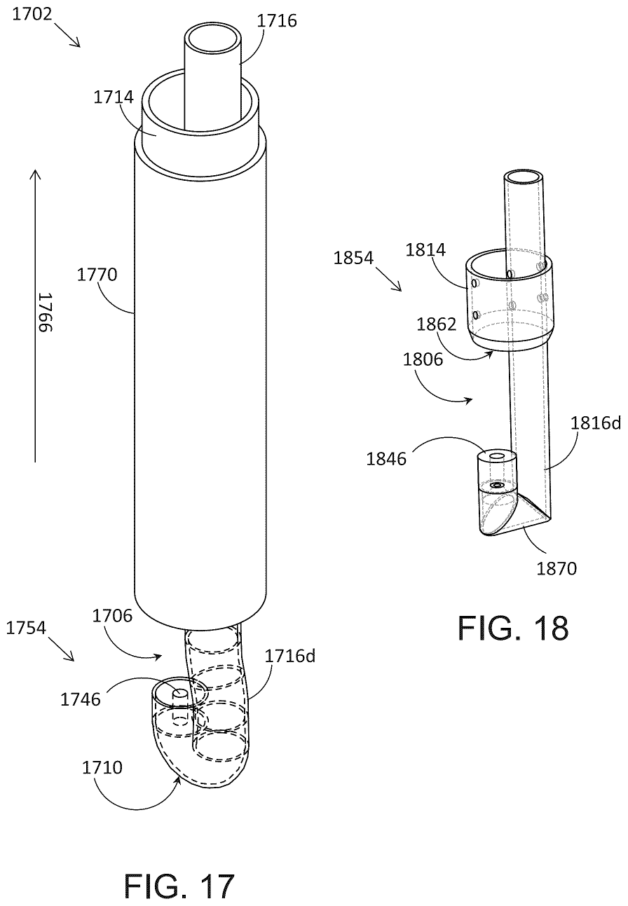

FIG. 17 is a simplified schematic of a portion of an arm 1702 including a tip 1754, according to some embodiments of the present disclosure.

In some embodiments, arm 1702 having a distal tip 1754 and portions extending in a proximal direction 1766 from tip 1754 includes a supply conduit 1716 extending through an extraction conduit 1714. Optionally, a distal portion 1716d of supply conduit 1716 extends out of (e.g., out of a distal end of) extraction conduit, where tip 1754 portion(s) are proximal of a gap 1706 between a nozzle outlet 1746 of supply conduit 1716 and extraction conduit 1714. In some embodiments, a structure forming nozzle 1746 is sized to fit into supply conduit 1716d. In some embodiments, arm portions proximal of gap 1706 are optionally surrounded by an outer sheath 1770.

In some embodiments, refraction of fluid supplied (e.g., by a supply conduit 1716, 1716d), prior to jetting across a gap 1706, is provided by bending of a distal portion of supply conduit 1716d. In some embodiments, bending of supply conduit 1716d forms a blocker 1710. In some embodiments, the bent portion of supply conduit 1716 is flexible, a potential benefit being low forces between blocker 1710 and adjacent tissue. In some embodiments, supply conduit 1716d is bendable and/or deflectable, for example, enabling a user to position gap 1706 and/or a jetting zone with respect to tissue; e.g., with reduced need to move arm portion(s) proximal of gap 1706.

FIG. 18 is a simplified schematic of a portion of a tip 1854, according to some embodiments of the present disclosure.

In some embodiments, tip 1854 includes one or more features of tip 1754 of FIG. 17 and/or is connected to arm portion(s) proximal of a gap 1806, proximal arm portion(s), in some embodiments, as described regarding FIG. 17.

In some embodiments, a distal portion 1870 of a supply conduit 1816d extends distally of gap 1806 between a supply conduit outlet 1846 and an inlet 1862 of an extraction conduit 1814. In some embodiments, supply conduit distal portion 1870 is rigid, a potential benefit being the ability to cut and/or move tissue using the distal end of the tip.

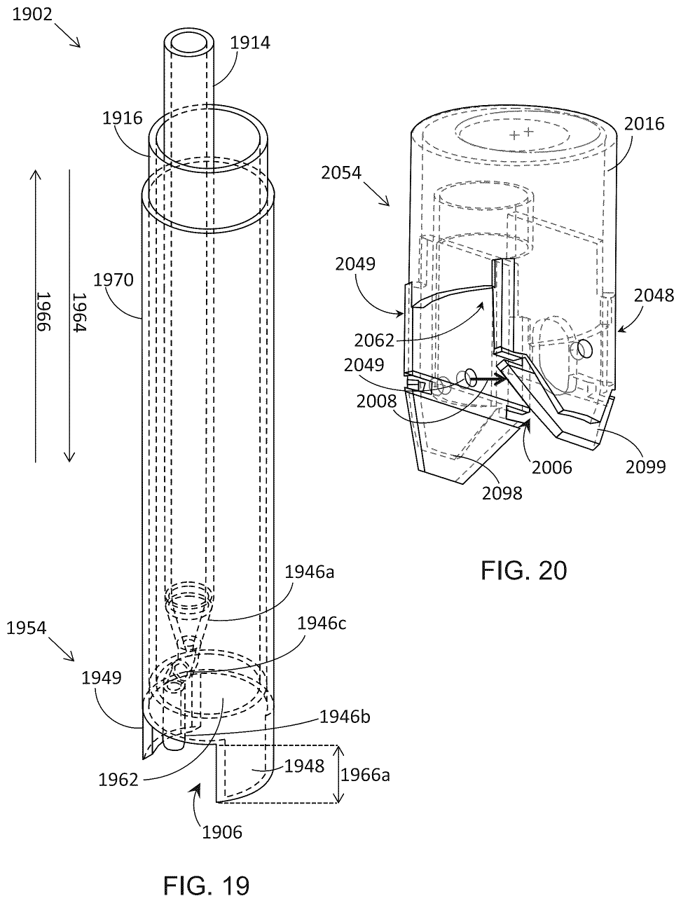

FIG. 19 is a simplified schematic of a portion of an arm 1902 including a tip 1954, according to some embodiments of the present disclosure. Arrow 1964 indicates a proximal-to-distal direction, and arrow 1966 indicates a distal-to-proximal direction.

In some embodiments, fluid delivered to a supply outlet 1946b transversely (e.g., with respect to a longitudinal axis of an arm distal end attached to tip 1954) across a space 1906 between sides 1948, 1949, of tip 1954, each extending distally by a distance of height 1966a. Potentially, transverse flow (or flow in a direction which includes a transverse component) enables gradual and/or gentle tissue erosion by the flow.

In some embodiments, a gap 1906 in tip 1954 is in a direction across a distal end of tip 1954. For example in a direction including a component perpendicular to a direction of elongation of one or more tubular components of arm 1902 and/or tip 1954; e.g., direction of elongation of one or more of a supply conduit 1914, an extraction conduit 1916, and an outer sheath 1970. Optionally, tip 1954 lacks a blocker.

In some embodiments, fluid is jetted across gap 1906 in a direction including a component perpendicular to a direction of elongation of one or more tubular components of arm 1902 and/or tip 1954; e.g., direction of elongation of one or more of a supply conduit 1914, an extraction conduit 1916, and an outer sheath 1970.

In some embodiments, nozzle outlet 1946b is located in a wall of supply conduit 1914. Optionally, a portion of supply conduit 1914 proximal to nozzle outlet 1946b (e.g., a distal portion of supply conduit 1914) is narrowed (e.g., by a narrowing 1946a) and/or repositioned with respect to extraction conduit 1916 (e.g., by conduit portion 1946c). In some embodiments, narrowing 1946a includes one or more features and/or fulfils one or more functions as described regarding narrowing 1458 of FIG. 14. In some embodiments, repositioning 1946c positions outlet 1946b closer to an edge of gap 1906 and/or extraction conduit 1916 potentially increasing an extent of a jetting region extending across tip 1954; e.g., between cover portions 1949, 1948. In some embodiments, jetted fluid and/or eroded debris is extracted through an extraction inlet 1962 to extraction conduit 1916.

FIG. 20 is a simplified schematic of a tip 2054, according to some embodiments of the present disclosure.

In some embodiments, fluid delivered to a supply outlet 2049 flows 2008 transversely (e.g., with respect to a longitudinal axis of an arm distal and attached to tip 2054) across a space 2006 between sides 2048, 2049a, of tip 2054. In some embodiments, space 2006 has dimension(s) as described regarding gaps and/or openings of other tips described in this document. Potentially, transverse flow (or flow in a direction which includes a transverse component) enables gradual and/or gentle tissue erosion by the flow 2008. In some embodiments, sides 2048, 2049a include portions of an extraction conduit 2016 which extend distally with spaces 2006 between sides 2048, 2049a. In some embodiments, coupling of outlet 2049 to side 2049a is by a plate 2098. Optionally, plate 2098 provides mechanical strength to (e.g., extraction conduit portion(s) of) side 2049a and/or holds a supply conduit (not shown) in position to be fluidly connected to outlet 2049.

In some embodiments, plates 2098, 2099 include tapered ends, a potential advantage being increased pressure at the ends when the tip is pushed into tissue, potentially aiding insertion and/or cutting of tissue at the distal end of tip 2054.

In some embodiments, the flow 2008 is diffused by interaction with a second side 2048 which opposes the fluid flow. In some embodiments, jetted fluid and/or eroded tissue is extracted through extraction conduit 2016; e.g., through an extraction conduit inlet 2062. In some embodiments, edges of side(s) 2048, 2049a enable application of force with the tip; e.g., to cut and/or deflect tissue.

### Examples of Nozzles

FIGS. 21A-21C are simplified schematic sectional views of nozzles 2146a-2146c, according to some embodiments of the present disclosure.

In some embodiments, nozzles 2146a-2146c include inner shapes and/or dimensions selected to narrow the fluid flow therethrough (increasing its velocity in consequence), and/or to provide a stable flow of fluid jetted from the nozzle outlet at a distal end of the nozzle. The different nozzles have, in particular, different narrowing slopes and/or different distances along which they narrow. The jetting outflow directions correspond to the directions of arrows 2166a-2166c. These arrows are designated proximal-distal in orientation, although the nozzle optionally is pointed “backwards” to jet in a proximal direction of the device overall, or in another orientation.

Dimensions illustrated on the figures are examples; the scale unit is millimeters. For example, a total length of a nozzle 2146a-2146c is restricted to 1-5 mm, or 1-3 mm, or about 2.5 mm, or lower or higher or intermediate lengths or ranges. The restricted length, in some embodiments, reduces an extent of a portion of the tip which is rigid.

Reference is now made to FIG. 21A. In some embodiments, nozzle 2146a has a first portion 2102a defining a lumen 2106a out of which fluid is jetted. In some embodiments, lumen 2106a is fluidly interconnected with a narrowing lumen defined by second nozzle portion 2104a. In some embodiments, lumen 2106a has a constant cross sectional area and/or shape along length 2108a. In some embodiments, lumen 2106a is defined by a tubular channel insert held within first nozzle portion 2102a. In some embodiments, the lumen of second nozzle portion 2104a has decreasing cross sectional extent moving in a proximal-distal 2166a direction along a length 2110a of second portion 2104a. In some embodiments, inner walls 2112a of second portion 2104a have constant angled slope; e.g., forming a cone shape (with a top of the cone shape truncated by a portion of lumen 2106a defined by first portion 2102a). Outer chamfer 2116a is optionally provided to assist in the fitting of a nozzle to fluid supply tubing.

Reference is now made to FIG. 21B, which, in some embodiments, includes one or more features of FIG. 21A (in FIGS. 21A-21C, reference character labels which are the same except for the final letter correspond, apart from dimensional differences as noted in millimeter measurements on the figure, and/or described in the text). Correspondences include, for example, a lumen 2106b, a first nozzle portion 2102b, a second nozzle portion 2104b, second portion inner walls 2112b, a first portion length 2108b, and a second portion length 2110b.

In some embodiments, nozzle 2146b of FIG. 21B, for a same given overall length (first portion length 2108b+second portion length 2110b) of the nozzle, has a longer second portion 2102b (than that of FIG. 21A), a shorter second portion 2104b and less highly sloped inner walls 2112b of second portion 2104b.

Reference is now made to FIG. 21C, which, in some embodiments, includes one or more features of FIG. 21A; including, for example, a lumen 2106c, a first nozzle portion 2102c, a second nozzle portion 2104c, second nozzle portion inner walls 2112c, a first nozzle portion length 2108c, and a second nozzle portion length 2110c.

In some embodiments, nozzle 2146c of FIG. 21C, has the same lengths as nozzle 2146a, of FIG. 21A, however, the inner slope 2112c is lower (less angled relative to the proximal-distal axis) than that of FIG. 21A.

In some embodiments, an angle of the slope of inner walls 2112a-2112c is 10-45°, or 15-40°, or 20-40°, or about 20°, or about 30°, or about 37°, or lower or higher or intermediate ranges or angles.

### Experimental Results

Tip designs were tested by jetting into a simulated clot composition of 1:39 saline to citrated blood, with 25 mM CaCl₂.

Tests included for different ambient pressures (e.g., 3-6 bar), jetting flow rates (e.g., 0.1-0.2 liters/min), suction flow rates (0.08-0.3 liters/min), and for different speeds of lateral movement of the tip through the simulated clot while jetting.

The removal of material was then evaluated visually; for example, to check shape and/or extent of material removal.

Experiments using a tip including features according to the tip illustrated in FIG. 10 produced a smooth edged material removal shape with an extent similar to an extent of the tip (e.g., width of the hollow of removed material was within 20% of the size of the tip).

Experiments using a tip including features according to the tip illustrated in FIG. 14 produced a smooth edged material removal shape with an extent wider than that of the tip (e.g., within 50% of the size of the tip).

Experiments using a tip including features according to the tip illustrated in FIG. 11 produced a material removal shape with jagged edges.

Experiments using a tip including features according to the tip illustrated in FIG. 12 produced a material removal shape with jagged edges.

Experiments using a tip including features according to the tip illustrated in FIG. 20 produced a material removal shape with jagged edges, even with low flow rate of 0.19 l/min and a low suction rate of 0.15-0.2 l/min.

Experiments using a tip including features according to the tip illustrated in FIG. 18 produced a material removal shape with jagged edges.

### General

It is expected that during the life of a patent maturing from this application many relevant robotic positioning technologies will be developed; the scope of the term robotic positioning is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, to the same extent as if each individual publication, patent or patent application was specifically and individually noted when referenced that it is to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

1. A device for selective tissue removal comprising a tip having:
a housing sized and shaped to enter a cranial cavity through an aperture 5mm in diameter;
a nozzle positioned to jet fluid supplied to said nozzle as a fluid jet disposed within and restricted by a volume defined by said housing; and
an opening in said housing to said fluid jet, sized and shaped for said fluid jet to erode tissue adjacent to said volume.

2. The device according to claim 1, wherein said opening is sized and shaped to allow said tissue to intrude into said opening.

3-4. (canceled).

5. The device according to claim 1, wherein said opening is to a side of said fluid jet.

6. The device according to claim 1, wherein a portion of said fluid jet flows alongside said opening.

7. The device according to claim 1, wherein said jet impinges on an inner surface of said housing.

8. The device according to claim 1, wherein said fluid jet is in a direction alongside or away from said opening, said fluid jet not impinging directly onto tissue outside said housing.

9. The device according to claim 1, wherein:
said tip comprises a nozzle and a blocker; and
said fluid jet is jetted through said nozzle and travel of said fluid jet is restricted by said blocker.

10. The device according to claim 9, wherein said tip has a proximal and a distal end, the blocker is positioned distal of said nozzle, and disrupts flow of jetted fluid distal of said blocker.

11-14. (canceled).

15. The device according to claim 1, comprising a fluid supply conduit which supplies fluid to said nozzle, wherein said fluid supply conduit extends in a proximal-distal direction and includes a turn at said tip prior to fluidly connect to said nozzle to supply fluid for said fluid jet.

16. The device according to claim 15, wherein said device comprises a suction conduit having suction inlet disposed at said tip for extraction of fluid and eroded tissue.

17. The device according to claim 1, wherein said device comprises an arm hosting said tip at a distal portion thereof, wherein said arm is bendable at one or more joints; and
wherein said device includes one or more actuator configured to bend said arm at said one or more joints.

18-19. (canceled).

20. The device according to claim 17, wherein:
said device comprises a suction conduit having suction inlet disposed at said tip for extraction of fluid and eroded tissue; and
said fluid supply conduit and said suction conduit extend through said arm.

21. The device according to claim 20, wherein said fluid supply conduit extends through a lumen of said suction conduit.

22. The device according to claim 1, comprising a second nozzle positioned to jet fluid supplied to said second nozzle as a second fluid jet disposed within and restricted by said volume defined by said housing; and
wherein said housing comprises a second opening, sized and shaped for said second fluid jet to erode tissue adjacent to said volume.

23-25. (canceled).

26. A method of removal of target tissue comprising:
positioning an opening to a housing adjacent to the target tissue, which housing hosts an outlet to a fluid supply; and
supplying fluid to said outlet to provide a fluid flow within said housing which fluid flow interacts, through said opening, with said target tissue to erode said target tissue.

27. The method according to claim 26, wherein said outlet comprises a nozzle, which produces a fluid jet upon said supplying.

28. The method according to claim 27, wherein said fluid jet is at least partially enclosed by said housing.

29. (canceled).

30. The method according to claim 28, comprising blocking a path of said water jet.

31. The method according to claim 26, comprising removing eroded tissue by suctioning eroded tissue and jetted fluid.

32. (canceled).

33. The method according to claim 26, where said eroding is of a portion of said target, and said method comprises repositioning said opening to continue to erode target tissue,
wherein said target tissue is tissue within a cranial cavity.

34-37. (canceled).
